(19) United States
(12) Patent Application Publication
(10) Pub. No.: US 2013/0273181 A1
(43) Pub. Date: Oct. 17, 2013

(54) METHODS AND COMPOSITIONS FOR DIAGNOSING AND TREATING MUSCLE MYOPATHY DISORDERS

(71) Applicant: The Regents of the University of Colorado, a body Corporate, Denver, CO (US)

(72) Inventors: MARTIN GARTZ HANSON, AURORA, CO (US); LEE NISWANDER, CASTLE ROCK, CO (US)

(21) Appl. No.: 13/789,612

(22) Filed: Mar. 7, 2013

Related U.S. Application Data
(60) Provisional application No. 61/608,040, filed on Mar. 7, 2012.

(51) Int. Cl. A61K 31/64; C12Q 1/68

(52) U.S. Cl. CPC A61K 31/64; C12Q 1/686
USPC 424/722; 435/6.12; 514/593

(57) ABSTRACT

Embodiments herein generally relate to methods and compositions for diagnosing and treating congenital muscle diseases (e.g. muscle myopathy disorders). In certain embodiments, methods and compositions generally relate to treating a subject having a muscle myopathy disorder with a dietary supplement and/or sulfonylurea agent.

### RELATED APPLICATIONS

This application is a U.S. Non-Provisional Application that claims priority to U.S. Provisional Application No. 61/608,040 filed on Mar. 7, 2012. The provisional application is incorporated herein by reference in its entirety for all purposes

### FIELD

Embodiments of the present invention generally relate to methods and compositions for diagnosing and treating congenital muscle diseases (e.g. muscle myopathy disorders) in a subject. Certain embodiments concern treating a subject known to have a muscle myopathy disorder with a supplement or a pharmaceutical agent to modulate potassium levels in the subject. Other methods concern diagnosing a subject with a muscle myopathy disorder by assessing biomarker expression levels as an indicator of the muscle myopathy disorder; and, treating the disorder in the subject.

### BACKGROUND

Muscle myopathies are muscle diseases that in part reduce functionality of myofibers causing weakness and, in some instances, death. Congenital myopathies are muscle diseases present at birth (congenital) that result from genetic defects in your muscle development. Congenital myopathies may cause a subject to have muscle weakness and breathing and eating problems. There are several types of congenital muscle myopathies that include nemaline myopathy, one of the most common congenital myopathy. Infants usually have problems breathing and with feeding. Another is myotubular myopathy, a rare disease that only affects boys. Weakness and floppiness are so severe that a mother may notice reduced movements of the baby in her womb during pregnancy. There are usually significant breathing and swallowing difficulties; many children do not survive infancy. Osteopenia (weakening of the bones) is also associated with this disorder. Other muscle myopathies include centronuclear myopathy, multi-minicore disease which has several subtypes, congenital fiber-type disproportion myopathy, and hyaline body myopathy

One muscle myopathy is referred to as central core disease (CCD) an inherited and currently incurable congenital muscle myopathy characterized by hypotonia and motor development delay with weakness in skeletal muscles. CCD varies among children with regard to the severity of problems and the degree of worsening over time. Usually, there is mild floppiness in infancy, delayed milestones, and moderate limb weakness, which do not worsen much over time. Children with CCD may have life-threatening reactions to general anesthesia. A hallmark pathology of CCD is muscle devoid of mitochondria within the central region. In normal muscle, nuclei are located at the periphery of the myofibers and the mitochondria reside throughout the myofiber. Muscle biopsies of CCD patients show central nuclei, as well as disorganized areas in the center of the myofiber called “Cores” that lack mitochondria and are devoid of metabolic activity, reflective of cellular damage.

### SUMMARY OF THE INVENTION

Certain embodiments concern using one or more biomarkers to diagnose muscle myopathy disorders. In other embodiments, compositions and methods concern treating a subject with a muscle myopathy disorder. In yet other embodiments, the muscle myopathy disorder can be any muscle myopathy disorder other than a cardiac disorder. In accordance with these embodiments, certain muscle myopathies can be associated with mutations or change in levels of certain biomarkers. An example of one of these biomarkers can be ryanodine receptor type 1 (RyR1). Mutations in RyR1 are linked to certain muscle myopathy disorders that include, but are not limited to, central core disease (CCD), multi mini core disease, hypokalemic periodic paralysis and malignant hyperthermia. Mutations in RyR1 can lead to the expression of mutant forms of RyR1 proteins or deletion of the protein altogether. In certain embodiments disclosed herein, a sample can be obtained from a subject and RyR1 can be analyzed for mutations in order to assess presence or severity of a muscle myopathy in the subject.

In addition, other diagnostic markers for assessing presence or progression of muscle myopathy can include, but are not limited to, assessing molecular levels of one or more of, Kcnj8 (which encodes the Kir6.1/KATP6.1 protein), Prkaa1 (which encodes the AMPK protein, AMP-activated protein kinase), Abcc8 (which encodes ATP-binding cassette transporter sub-family C member 8), Na/K-ATPase α1 and/or Na+, K+-ATPase α2. In certain embodiments, samples can be obtained from a subject and prepared for analysis of presence for or levels of two or more of these biomarkers in order to assess presence or progression of a muscle myopathy in the subject. In other embodiments, all of these biomarkers can be analyzed for level of expression in order to assess presence or progression of a muscle myopathy condition.

Other embodiments provide for compositions, methods and uses of biomarkers diagnose a muscle myopathy in a subject and then to treat the muscle myopathy disorder using compositions disclosed herein. For example, in certain embodiments, biomarker levels can be assessed in a muscle sample obtained from a subject in need thereof, in order for a health professional to assess need for treatment of the subject for the muscle myopathy disorder. In accordance with these embodiments, a subject having modulated Kcnj8, Na+, K+-ATPase α1, Prkaa1, Abcc8 and/or Na+, K+-ATPase α2 expression levels compared to a control level is likely to have or to develop a muscle myopathy disorder. These subjects can then be candidates for treatment for the muscle myopathy disorder. In other embodiments, a subject having RyR1 mutations and modulated Kcnj8, Na+, K+-ATPase α1, Prkaa1, Abcc8 and/or Na+, K+-ATPase α2 expression levels compared to a control level is likely to develop or have a muscle myopathy disorder. In certain embodiments, the expression level of these genes is increased relative to a control sample (e.g. a subject not having a muscle myopathy disorder). Further, a subject identified as having RyR1 mutations diagnosed with or suspected of developing a muscle myopathy disorder can be treated with supplemental potassium which can alleviate symptoms as well as treat the disorder. Alternatively, a subject with RyR1 mutations diagnosed with or suspected of developing a muscle myopathy disorder can be treated with one or more sulfonylurea agents (e.g. Glyburide).

In other embodiments, a subject can be diagnosed with CCD by assessing mutations in RyR1, for example by assessing presence of heterozygous or homozygous mutations in the RyR1 gene. A heterozygous mutation in RyR1 display clinical and pathological hallmarks of CCD and homozygous RyR1 displays complete loss of movement in the subject. These assessments may be performed alone or coupled with assessing molecular levels of one or more of Kcnj8, Prkaa1, Abcc8, Na+, K+-ATPase α1 and/or Na+, K+-ATPase α2 in the subject. In accordance with these embodiments, a subject diagnosed with CCD can be treated using supplemental potassium which can alleviate symptoms as well as treat the disorder. Alternatively, a subject having CCD can be treated with one or more sulfonylurea agents (e.g. Glyburide).

Other embodiments can concern diagnostic kits for diagnosing and assessing treatment of muscle myopathy disorders in a subject. For example, a kit can be used to detect level of expression of biomarkers or changes in the levels of expression during a predetermined window of time in a subject. In accordance with these embodiments, a biomarker can include one or more of Kcnj8, Prkaa1, Na+, K+-ATPase α1 and Na+, K+-ATPase α2 alone or in combination with compositions for assessing mutations in RyR1 to diagnose and/or treat a muscle myopathy disorder in the subject. Levels of expression can be measured at an mRNA level and/or a protein level. In other embodiments, kits can be used by a health professional or laboratory to assess presence or absence of a muscle myopathy disorder in a subject. These disorders include, but are not limited to, central core disease (CCD), multi mini core disease, hypokalemic periodic paralysis and malignant hyperthermia. In one embodiment, the muscle myopathy disorder is CCD.

Some embodiments provide compositions and methods for treating a subject diagnosed with a muscle myopathy disorder. In accordance with these embodiments, any known treatment for these disorders can be used to treat a subject diagnosed with a disorder in combination with potassium supplements and/or sulfonylurea agents. In some embodiments, treatment can include dietary potassium supplements for treating the muscle myopathy disorders alone or in combination with other supplements. Dietary potassium doses can include those known by one skilled in the art as a recommended daily dose or at a safe dose higher than the recommended dose to rapidly alleviate symptoms of the affected subject.

Other embodiments concern generating a non-human animal model, for example, an acceptable mouse model for studying CCD and treatments for muscle myopathy disorders. In accordance with these embodiments, a mouse RyR1 model in which homozygous RyR1 mutant muscle lacks contractility and heterozygous animals display clinical and pathological hallmarks of CCD. Certain embodiments disclosed herein concern testing pharmaceutical and dietary agents using the RyR1 mouse model. In addition, genes in the mouse model can be studied in order to assess whether a target gene is linked to CCD.

### BRIEF DESCRIPTION OF THE DRAWINGS

The following drawings form part of the present specification and are included to further demonstrate certain embodiments herein. The embodiments may be better understood by reference to one or more of these drawings alone or in combination with the detailed description of specific embodiments presented.

FIGS. 1a-1c represents a mouse model and control mouse (1a) illustrating a muscle myopathy of certain embodiments disclosed herein; mouse model images that represent different mutations in the Ryr1 gene and a control mouse (far left) (1b) and 1c, illustrating control and mutated Ryr1 sequences of certain embodiments disclosed herein.

FIGS. 2a-2i: FIGS. 2a-2b represent exemplary graphs illustrating Ca²⁺ release (2a) and Δ F-V relationships (2b) elicited by electrical stimulus in normal mice and a disease mouse model of certain embodiments described herein. FIG. 2c represents an exemplary histogram illustrating muscle contractions induced by electrical stimulus in control mice and a disease mouse model before and after receiving certain treatment. FIG. 2d represents an exemplary diagram plotting Ca²⁺ transients induced by a pharmacological stimulation in normal mice and a disease mouse model. FIG. 2e is an exemplary histogram demonstrating half-rise time of Ca²⁺ transients elicited by a pharmacological stimulation in normal mice, a disease mouse model and mutant control mice. FIG. 2f represents exemplary data demonstrating muscle contractions induced in normal mice and a disease mouse model before and after treatment with certain chemicals. FIGS. 2g-2i present exemplary diagrams illustrating muscle contractility in normal mice, a disease mouse model and mutant control mice induced by various pharmacological stimulus of certain embodiments disclosed herein.

FIGS. 3a-3j FIGS. 3a-3b represent exemplary histograms of muscle strength demonstrated by normal mice and a disease mouse model at different ages (juvenile to adult). FIGS. 3c-3j represent exemplary images illustrating pathology and histology of muscle sections from a disease mouse model compared to a normal mice on various diets.

FIG. 4 represents an exemplary graph demonstrating muscle contractility in a disease mouse model induced by electrical stimulation in the presence of chemicals and pharmacological stimulus of certain embodiments provided herein.

FIGS. 5a-5s: FIGS. 5a-5c represent exemplary histograms illustrating muscle strength (5a and 5b) and certain histopathological analysis (5c) of muscle samples from normal mice and a disease mouse model treated on different diets (at different ages) and pharmacological stimulus disclosed in certain embodiments herein. FIGS. 5d-5s represent histological and pathological images illustrating muscle sections from a disease mouse model and normal mice receiving different supplements of certain embodiments herein.

FIGS. 6a-6e represent (6a) expression levels of various genes of interest compared to controls; (6b-5e) are histogram plots representing expression levels of various genes of interest in normal and diseased mice on various supplements of certain embodiments disclosed herein.

FIGS. 7a-7b represent exemplary histological images of muscle sections from normal mice (7a) and a disease mouse model (7b) treated with certain pharmacological stimulation disclosed in certain embodiments provided herein.

FIGS. 8a-8e; FIGS. 8a-8d represent exemplary graphs demonstrating membrane potential shift observed by changes of potassium concentrations in muscles from normal mice and a disease mouse model receiving various pharmaceutical agents and on various supplementary diets; and FIG. 8e illustrated a time course for administration of a pharmacological agent and affects on potential in a normal and diseased mouse of certain embodiments provided herein.

### DETAILED DESCRIPTION

The following drawings form part of the present specification and are included to further demonstrate certain embodiments of the present invention. The embodiments may be better understood by reference to one or more of these drawings in combination with the detailed description of specific embodiments presented herein.

Certain embodiments of the present invention concern using biomarkers to diagnose muscle myopathy disorders in a subject. Myopathies decrease functionality of the myofiber causing muscle weakness and sometimes death. Further, certain muscle myopathies contemplated herein are associated with mutations in ryanodine receptor type 1 (RyR1) that include, but are not limited to, central core disease (CCD), multi mini core disease, hypokalemic periodic paralysis and malignant hyperthermia. CCD is an incurable congenital myopathy. RyR1 mutations can lead to the expression of mutant forms of RyR1 proteins or deletion of the protein which correlated with certain muscle myopathies. In addition, RyR1 mutations in muscle bring about diseases such as multi mini core disease (MMD), hypokalemic periodic paralysis and malignant hyperthermia (MH).

In certain embodiments, a mouse RyR1 model in which homozygous mutant muscle lacks contractility and heterozygous animals display clinical and pathological hallmarks of CCD is derived. Transcriptional and post-transcriptional dysregulation of three mechanisms required for potassium transport is identified resulting in increased resting membrane potential. In other embodiments, RyR1-dependent muscle contractions can be restored by pharmacological rectification of the membrane potential and by increasing potassium signaling either by diet or FDA-approved drugs. Application of these treatments results in reversal of the muscle weakness and CCD pathology. In other embodiments, regulators of potassium homeostasis are identified as biomarkers of disease the mouse model as well as in human patients having CCD. These studies support that regulators of potassium homeostasis can serve as diagnostic biomarkers and possible therapeutic targets in human patients with CCD.

### Ryr1 Gene

Ryr1 gene in muscle is well characterized with respect to their mechanisms for epolarization and voltage gated channel induced release of intracellular calcium stores. Muscle movement is produced through excitation-contraction coupling (E-C coupling), which is elicited by t-tubule epolarization produced by the opening of sodium channels (Nav 1.4; gene name Sen4a). Depolarization triggers the opening of the dihydropyridine receptor (L-type calcium channel DHPR; encoded by the gene called Caenals or Cavl.J, which stimulates the coupled response of ryanodine receptor RyR1 in the sarcoplasmic reticulum (SR). RyR1 protein acts as a calcium release channel to flood the myofiber with calcium, which initiates the contraction in the skeletal muscle to permit locomotion. In CCD and MMD myopathies, the mutant RyR1 protein may leak Ca2+ from the SR or the channel portion of RyR1 is uncoupled, thus preventing Ca2+ release.

Myopathies due to RyR1, Caenals or Sen4a mutations are associated with hypokalemia, or low serum potassium. Hypokalemia can occur due to defects in potassium homeostasis, which is regulated by K+-transporting proteins that serve to maintain the resting membrane potential, protect against fatigue in the muscle, and preserve the E-C coupled release of internal calcium stores. When muscles are at rest, Na+, K+ ATPase is essential for maintaining the equilibrium potential of Na+ and K+ ions through ATP hydrolysis and phosphorylation of the Na+, K+-ATPase a1 subunit by serine/threonine kinases. In excitable cells such as muscle, Na+, K+-ATPase helps to repolarize the membrane to a resting state after an action potential. Failure to repolarize the membrane potential can lead to fatigue and stress to the muscle.

During fatigue or stress to the muscle, two types of K+-transporting channels, KJR2.1 and KATP, participate in setting the t-tubule and myofiber resting membrane potentials. The inward rectifying potassium channel (Kcnj2; KJR2.1) participates in the reuptake of K+ after an action potential. KATP channels buffer potassium during metabolic stress or fatigue. During strenuous muscle activity, ATP stores are depleted. In response, KATP channels (Kcnj11; KATP6.2) open to allow the efflux of potassium into the t-tubule and interstitial space and KATP channels in mitochondria (Kcnj8; KATP6.1) open to increase ATP production. Thus, some embodiments herein concern analysis of disruption of these K+-transport mechanisms that could alter calcium release from the SR and energy production, leading to lack of contractility and muscle weakness in a subject.

Mutations of Ryr1 gene in muscle myopathy disorders are associated with altered pathway of potassium transport through regulations of Na+, K+-ATPase, KIR and KATP channels. In some embodiments, changes of potassium level in serum can modulate muscle weakness caused by mutant RyR1, and affect the expression levels of Kcnj8, Prkaa1 and Na+, K+-ATPase α2. In accordance with these embodiments, potassium supplement can reverse muscle weakness and pathology of muscle myopathy disorders including, but not limited to, central core disease (CCD). The expression of Kcnj8, Prkaa1 and Na+, K+-ATPase α2 can be restored to normal levels after administration of potassium supplement (Table 1). In other embodiments, inhibitors are used to modulate serum potassium level. The inhibitors comprise, but are not limited to angiotensin-converting enzyme. In other embodiments, drugs are used to modulate muscle myopathy disorders, including but not limited to, CCD. The drugs concerned herein include, but are not limited to, sulfonylurea class of anti-diabetic agents including but not limited to, Glibenclamide and 5-hydrodecanoic acid (5HA).

Decreased mitochondria activity is associated with muscle myopathy disorders. In certain embodiments, potassium supplement influences mitochondrial activity and restores KATP6.1 expression level. In accordance with these embodiments, KATP6.1 may be a marker for decreased mitochondrial activity in muscle myopathy disorders including, but not limited to CCD. Thus, analysis for the muscle myopathy may lead to treatment with known commercially available treatments alone or in combination with potassium replenishment.

### Mouse Model

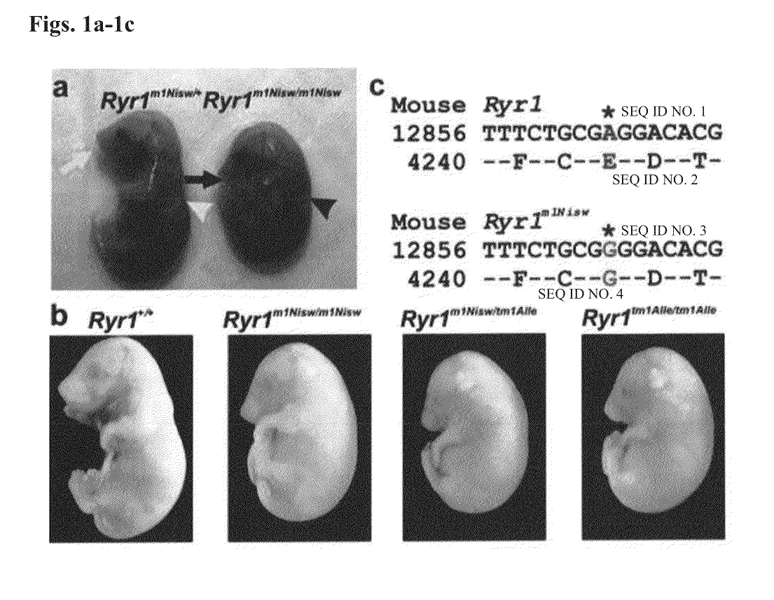

In other embodiments, a mouse model of use for studying CCD was developed. Using forward genetic screening in mice, a recessive mutation was identified in which homozygous mutant embryos displayed kyphosis (hunchback), carpoptosis (wrist drop), severe hypotonia (see Examples), and die shortly after birth without breathing or moving. Meiotic recombination mapped the mutation to a small region of chromosome 7 containing the Ryr1 gene and a complementation cross with a Ryr1 null allele (Ryr1^{tm1Alle}) showed failure to complement (see Examples). Sequencing identified a missense mutation in exon 93 of Ryr1 that changes a glutamic acid (4242) to a glycine (see for example, FIG. 1) and is outside of the channel region of RyR1. This allele is referred to as Ryr1^{m1Nisw}. The mutant protein is present in muscle and myotube cultures, whereas no protein is detected in Ryr1^{tm1Alle} null myotubes. Thus, RyR1^{m1Nisw} mutant protein is present but not capable of eliciting locomotor activity. Assays using fluorescent Ca²⁺ indicator, electrical stimulation, and RyR1 agonist to measure release of SR Ca²⁺ stores and muscle contraction indicated that Ryr1^{m1Nisw} encodes a hypomorphic protein that is uncoupled and fails to respond to direct change in membrane potential, but that Ca²⁺ stores are not depleted, although the rate of Ca²⁺ release is significantly impaired. Under physiological conditions, RyR1^{m1Nisw} protein is non-functional in skeletal muscle. Ryr1^{m1Nisw/+} heterozygous mice did not display muscle wasting but demonstrated muscle weakness. One month, two month, and one year old RyR1^{m1Nisw/+} mice showed a significant decrease in grip strength on a wire hanging task compared to Ryr1^{+/+} mice (see Examples) but they did not show MH (see methods). Thus, heterozygous mutant mice show muscle weakness, one of the most poignant clinical manifestations of CCD.

In other embodiments, it was observed that RNA and protein expression of Kcnj8, encoding the K_{ATP}6.1 channel, was downregulated in Ryr1^{m1Nisw/m1Nisw} and Ryr1^{m1Nisw/+} muscle and Kcnj11, the t-tubule/plasma membrane K_{ATP}6.2 channel, was downregulated in Ryr1^{m1Nisw/m1Nisw} embryonic muscle where restoration of gene expression alleviates symptoms of the myopathy. Further, potassium transport activity of K_{ATP} channels is positively regulated by AMP-activated protein kinase (AMPK). The catalytic transcript of AMPK (encoded by Prkaa1) and AMPK protein levels were significantly downregulated in the mouse model Ryr1^{m1Nisw/+} and Ryr1^{m1Nisw/m1Nisw} E18.5 muscle, although transcripts were upregulated in adult Ryr1^{m1Nisw/+} muscle. In other embodiments, sulfonylurea receptors (SUR), ATP-binding cassette (ABC) transporters, were assessed for correlation with the myopathy. SUR bind to and inhibit K_{ATP} channels. SUR1 (abcc8) was significantly upregulated in Ryr1^{m1Nisw/+} and Ryr1^{m1Nisw/m1Nisw} embryonic muscle and diseased human muscle samples supported this observation. It is noted that RyR1 increases ERK1/2 protein levels and ERK1/2 can regulate transcription and phosphorylate the major potassium transport protein, Na⁺, K⁺-ATPase, which maintains Na⁺ and K⁺ equilibrium at the membrane. ERK2 protein levels were modestly, yet significantly reduced in Ryr1^{m1Nisw/m1Nisw} muscle. The pump subunits Na⁺, K⁺-ATPase α1 and α2 were increased in Ryr1^{m1Nisw/m1Nisw} and Ryr1^{m1Nisw/+} embryonic muscle but, consistent with decreased ERK2 protein levels, ERK1/2 dependent phosphorylation of Ser-23 on Na⁺, K⁺-ATPase α1 was decreased in Ryr1^{m1Nisw/m1Nisw}. Na⁺, K⁺-ATPase α1 and α2 levels were both decreased in Ryr1^{m1Nisw/+} adult muscle. Thus, a number of genes and proteins that mediate potassium transport across the membrane are modulated in mutant muscle observed in the mouse model and in human samples. Inability to rectify resting membrane potential after an action potential and lack of excitability of the mutant muscle in the affected subject is due in part toaltered expression of these channels.

### Potential Treatments

Other embodiments concern altering serum potassium levels in a subject having CCD to restore modulated pathways of a CCD affected subject. In accordance with these embodiments, serum potassium levels can be modulated through diet or angiotensin-converting enzyme (ACE) inhibitors, for example. In order to assess affects of altering serum potassium levels, Ryr1^{m1Nisw/+} and Ryr1 mice were raised on 0.6% K (level of potassium in standard mouse chow, control) for four weeks (muscle weakness was observed, see Examples) and then fed either low 0.1% or high 5.2% potassium diet to provide hypokalemic or hyperkalemic conditions, respectively, or given the ACE inhibitor, Enalapril (0.02 mg/ml in drinking water), for an additional four weeks. Weakness in Ryr1^{m1Nisw/+} mice was clearly evident on 0.1% and 0.6% K diets with ˜50% reduction in grip strength and inability to hang onto the wire. Ryr1^{m1Nisw/+} mice administered Enalapril showed increased grip strength. Most dramatically, 5.2% K diets rescued and even increased grip strength and rescued muscle strength as assayed by hanging task in RyR1^{m1Nisw/+} mice compared to Ryr1^{+/+} mice. Therefore, potassium supplementation as disclosed herein rescues muscle weakness in affected subject, for example those linked to a RyR1^{m1Nisw} mutation.

In other embodiments, it was demonstrated that muscle of affected subjects provided a high potassium diet (e.g. 5.2% of intake) resembled control muscle (see Examples). In other embodiments, Enalapril (an angiotensin converting enzyme (ACE) inhibitor) demonstrated an improvement in muscle histology. Even after muscle dysfunction, muscle myopathies can be rescued with increased extracellular potassium as a supplement or as a pharmaceutical agent to increase potassium intracellularly. In certain embodiments, administration of a type II diabetes agent, glyburide, which selectively binds SUR1/2, by activating ATP-sensitive K⁺ channels, can rescue a subject from clinical and pathological manifestations of CCD (see Examples). These SURdirected agents can be used as an alternative treatment or in addition to supplemental potassium. Use of FDA-approved drugs known to alter SUR1/2 would not involve changing serum potassium levels in a subject as treated.

### Diagnosis

In certain embodiments disclosed herein, RyR1 may be analyzed for mutations in order to assess presence of a muscle myopathy in the subject. In addition, other diagnostic markers for assessing muscle myopathies can include, but are not limited to, assessing molecular levels of one or more of, Kcnj8 (which encodes the Kir6.1/KATP6.1 protein), Prkaa1 (which encodes the AMPK protein, AMP-activated protein kinase), Abcc8, Na+, K+-ATPase α1 and Na+, K+-ATPase α2 and comparing these expression levels to control levels of the genes, nucleic acids or proteins. In certain embodiments, two or more, three or more, four or more or five or more biomarkers can be analyzed for level of expression. In other embodiments, all five of these biomarkers can be analyzed for level of expression with or without analysis of mutations of an Ryr1 gene.

Certain embodiments concern analyzing expression levels of K_{ATP}6.1 protein and transcript expression in a subject having CCD before and after providing the subject with a high potassium diet. Other embodiments concern assessing expression levels of Prkaa1 transcripts in an affected subject on high potassium diets in order to measure restoration of normal, control levels. Yet other embodiments concern assessing expression levels of Abcc8 transcripts in an affected subject on high potassium diets in order to measure restoration of normal, control levels. Potassium supplementation is capable of rescuing some of the key proteins required for muscle activity in CCD muscle, suggesting that K_{ATP}6.1, Prkaa1, Abbc8, and Na⁺, K⁺-ATPase α1 and Na⁺, K⁺-ATPase α2 are biomarkers for CCD myopathy and recovery.

In addition, human studies confirmed the results observed in the mouse mode. Biomarkers isolated from RNA isolated from human skeletal muscles collected from CCD patients compared to control human samples without muscle disease. Patients 1 and 2 had RYR1 mutations identified while patients 3 and 4, did not carry mutations in RYR1. (See Examples) As shown in Supplemental Table 3, patients 1 and 2 showed a highly significant increase in expression of KCNJ8 (K_{ATP}6.1), PRKAA1, ABCC8 and ATPA1 (Na⁺, K⁺-ATPase α1). These data support the observation that muscle from human CCD patients with RYR1 mutations is sensitive to pathways that mediate potassium homeostasis. Patient 3 had significantly increased expression of KCNJ8, PRKAA1 and ABCC8 but a decrease in ATPA1. In patient 4, only ABCC8 was elevated, whereas KCNJ8 and ATPA1 were significantly decreased. These data could suggest that differences in these potassium homeostasis pathways may lead to differential sensitivities in CCD patients with RYR1 mutations compared to those with non-RYR1 mediated CCD. In certain embodiments, these diagnostic biomarkers can be used to assess efficacy and length treatment of CCD in an affected subject through modulation potassium signaling pathways. Thus, agents that increase calcium release or maintain potassium homeostasis can be used to reset the resting membrane potential to allow sustained muscle contractions to treat these conditions by alleviating symptoms and treating muscle myopathy conditions (e.g. CCD).

In other embodiments, compositions of use to treat a muscle myopathy condition contemplated herein can be a potassium-enriched diet alone or in combination with one or more sulfonylurea class of anti-diabetic agents. In other embodiments, as some diseases associated with RYR1 mutations, such as MH, are sensitive to potassium levels, a patients or biopsy samples would be tested for these sensitivities prior to potassium supplementation. Thus, both RYR1 and non-RYR1-linked myopathies (e.g. CCD) can be assessed using diagnostic biomarkers of disease identified herein and utilized to identify candidates for potassium modulation-based therapies.

Some embodiments provide compositions for treating a subject diagnosed with a muscle myopathy disorder in order to alleviate symptoms and/or treat the condition. In accordance with these embodiments, one or more sulfonylurea agents can be used. Sulfonylurea agents contemplated herein can include, but are not limited to, Carbutamide, Acetohexamide, Chlorpropamide, Tolbutamide, Tolazamide, Glipizide, Gliclazide, Glibenclamide (also known as Glyburide), Glibornuride, Gliquidone, Glisoxepide, Glyclopyramide, and Glimepiride. Some embodiments concern treating a subject diagnosed with a muscle myopathy disorder with an anti-diabetic sulfonylurea agent, Glyburide. Some embodiments provided herein concern a compositions including one or more sulfonylurea drugs and/or potassium supplements for treating a subject diagnosed with a muscle myopathy disorder. In accordance with these embodiments, such composition may include an anti-diabetic sulfonylurea agent (e.g. Glyburide) and potassium supplements. In certain embodiments, a sulfonylurea agent can be administered at about 0.1 mg to 10 mg to a subject daily, weekly or monthly or as recommended by a health provider.

| | |
|---|---|
| Category | Adequate Intake (AI) |

CHILDREN

| | |
|---|---|
| 0-6 months | about 400 mg/day |
| 7-12 months | about 700 mg/day |
| 1-3 years | about 3,000 mg/day |
| 4-8 years | about 3,800 mg/day |
| 9-13 years | about 4,500 mg/day |
| 14 years and up | about 4,700 mg/day |

ADULTS

| | |
|---|---|
| 18 years and up | about 4,700 mg/day |
| Pregnant women | about 4,700 mg/day |
| Breastfeeding women | about 5,100 mg/day |

High potassium diet or potassium supplements can include a supplement of about 1 to about 10% potassium of a consumable on a daily basis. In other embodiments, a potassium supplement can be about 0.1 gm to about 10 gms per day to restore muscle function in a subject having CCD. Other examples of dietary potassium are found below (World Health Organization (WHO)).

### Nucleic Acids

As described herein, an aspect of the present disclosure concerns isolated nucleic acids and methods of use of isolated nucleic acids. The term “nucleic acid” is intended to include DNA and RNA and can be either be double-stranded or single-stranded. In a preferred embodiment, the nucleic acid is a cDNA comprising a nucleotide sequence such as found in GenBank. In certain embodiments, the nucleic acid sequences disclosed herein have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically will be 10-20 nucleotides, but may be longer. Longer sequences greater than 50 even up to full-length, are preferred for certain embodiments.

In certain embodiments, it will be advantageous to employ nucleic acid sequences in combination with an appropriate means, such as a label, for determining hybridization. A wide variety of appropriate indicator means are available (i.e. fluorescent, radioactive, enzymatic or other ligands, such as avidin/biotin) that are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known which can be employed to provide a detection means visible to the human eye or spectrophotometrically, to identify specific hybridization with complementary nucleic acid-containing samples.

In general, it is envisioned that the hybridization probes described herein will not only be useful in solutions as in PCR, for detection of expression of corresponding genes but also in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under known conditions. Other embodiments concern RT-PCR or other methods known in the art for measuring nucleic acid levels.

### Protein Purification

Various methods for quantifying the degree of purification of the protein or peptide will be known to those of skill in the art in light of the present disclosure. These include, for example, determining the specific activity of an active fraction, or analysis by SDS/PAGE to identify the number of polypeptides in a given fraction. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will be dependent upon the particular assay technique chosen to follow the purification and whether or not the expressed protein or peptide exhibits a detectable activity.

Methods for purifying various forms of proteins are known. (i.e., Protein Purification, ed. Scopes, Springer-Verlag, New York, N.Y., 1987; Methods in Molecular Biology: Protein Purification Protocols, Vol. 59, ed. Doonan, Humana Press, Totowa, N.J., 1996). The methods disclosed in the cited references are exemplary only and any variation known in the art may be used. Where a protein is to be purified, various techniques may be combined, including but not limited to cell fractionation, column chromatography (e.g., size exclusion, ion exchange, reverse phase, affinity, etc.), Fast Performance Liquid Chromatography (FPLC), High Performance Liquid Chromatography (HPLC), gel electrophoresis, precipitation with salts, pH, organic solvents or antibodies, ultrafiltration and/or ultracentrifugation.

There is no general requirement that the protein or peptide always be provided in the most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

Any method for measuring protein expression (e.g. Abcc8 levels) known in the art is contemplated of use herein.

### Pharmaceutical Compositions and Routes of Administration

Aqueous compositions contemplated herein may include an effective amount of a therapeutic peptide, peptide construct, epitopic core region, stimulator, inhibitor, and the like, dissolved or dispersed in a pharmaceutically acceptable carrier or aqueous medium. Aqueous compositions of gene therapy vectors expressing any of the foregoing are also contemplated. The phrases “pharmaceutically or pharmacologically acceptable” refer to molecular entities and compositions that do not produce an adverse, allergic or other untoward reaction when administered to an animal, or a human, as appropriate.

Aqueous compositions contemplated herein may include an effective amount of the compound, dissolved or dispersed in a pharmaceutically acceptable carrier or aqueous medium. Such compositions can also be referred to as inocula. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

Biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

Pharmaceutical forms suitable for injectable use include sterile aqueous solutions or dispersions; formulations including for example, aqueous propylene glycol; and sterile powders for the extemporaneous preparation of sterile injectable solutions or dispersions. In all cases the form must be sterile and must be fluid. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A therapeutic agent can be formulated into a composition in a neutral or salt form. Pharmaceutically acceptable salts, include for example, acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. In terms of using peptide therapeutics as active ingredients, any method known in the art may be used.

Carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The term “unit dose” refers to physically discrete units suitable for use in a subject, each unit containing a predetermined quantity of the therapeutic composition calculated to produce the desired responses, discussed above, in association with its administration, i.e., the appropriate route and treatment regimen (e.g. 1.25 mg, 2.5 mg up to gram levels if by oral intake. The quantity to be administered, both according to number of treatments and unit dose, depends on the subject to be treated, the state of the subject and the protection desired. The person responsible for administration will, in any event, determine the appropriate dose for the individual subject.

The active therapeutic agents may be formulated within a mixture to comprise about 0.0001 to 1.0 milligrams, or about 0.001 to 0.1 milligrams, or about 0.1 to 1.0 or even about 10 milligrams per dose or so. Multiple doses can also be administered, daily, weekly, bi-weekly or monthly for example.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation.

Additional formulations which are suitable for other modes of administration include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1% 2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring.

In one embodiment, doses may be given once or more daily, weekly, monthly or yearly, or even once every 2 to 5 or more years. Persons of ordinary skill in the art may estimate repetition rates for dosing based on measured residence times and concentrations of the targetable construct or complex in bodily fluids or tissues. Following successful treatment, it may be desirable to have the patient undergo maintenance therapy to prevent the recurrence of the disease state, wherein the therapeutic agent is administered in maintenance doses, ranging from 0.01 ug to 100 mg per kg of body weight, once or more daily, to once every 5 years.

In another embodiment, a particular dose may be calculated according to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data.

### Kits

Some embodiments concern kits for compositions and methods disclosed herein. A kit can include, but is not limited to, one or more compositions in one or more containers or vessels containing reagents of use to measure muscle myopathy-linked genetic markers disclosed herein.

Kits contemplated of use herein can include a kit for medical applications or kits for research applications for further study.

### EXAMPLES

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

### Forward Genetic Screen for Locomotor Defects

In one exemplary method, target genes that are required for locomotor movements were identified by a rapid forward genetic screen developed in mouse embryos based on observations that embryonic day 18.5 (E18.5) embryos displayed reflex and complex coordinated movements. Following N-ethyl-N-nitrosourea (ENU) chemical mutagenesis and a three-generation cross to identify recessive mutations, E18.5 mouse embryos were screened for defects in flexor/extensor, righting-reflex, and axial movements. Various defects were observed in mutant embryos of mouse lines. For example, homozygous mutant embryos of line 1-4 displayed defects including kyphosis (hunchback), carpoptosis (wrist drop) and unresponsiveness during the locomotor screen due to severe hypotonia (see FIG. 1a). Then, meiotic recombination mapping was used to narrow the critical region containing the line 1-4 mutation to a three megabase region on proximal mouse chromosome 7. Bioinformatic analysis of this region revealed that the ryanodine receptor type 1 (RyR1) was involved in muscle contraction. In support of these findings, Ryr1 null mice (Ryr1^{tm1Alle/tm1Alle} alleles Ryr1^{tm1Tno/tm1Tno}) were to have a gross morphology similar to line 1-4 mutants. A further study using a complementation test cross between Ryr1^{tm1Alle} and line 1-4 showed failure to complement in resulted trans-heterozygous embryos with the same gross phenotype as RyR1^{m1Nisw/m1Nisw} mutants and RyR1^{tm1Alle/tm1Alle} nulls (FIG. 1b).

A Ryr1 gene contains 108 exons for a total coding region of 15,853 base pairs. Sequencing of the gene identified a missense mutation in exon 93 changes A to G, resulting in substitution of glutamic acid with glycine (E4242G) (FIG. 1c). Because the mutation was located outside the channel region of RyR1, line 1-4 was renamed Ryr1^{m1Nisw}. In order to determine whether mutant RyR1 protein was produced and stable, the muscle where RyR1 was predominant RyR present was examined with methods known in the art, for example, immunohistochemistry, where a pan RyR antibody, e.g. RyR antibody 34C, was applied to the wild type and mutant muscle samples. RyR immunoreactivity in puncta-like patterns of muscle fibers was observed in both heterozygous and homozygous mutant Ryr1^{m1Nisw} diaphragm muscle (data not shown). In myotube cultures, locomotor normal and mutant Ryr1^{m1Nisw} myotubes demonstrated detectable level of RyR protein, whereas no protein was detected in Ryr1^{tm1Alle} null myotubes (data not shown). Immunohistochemistry performed only with a secondary antibody served as a negative control. These data at least indicated that the RyR1^{m1Nisw} mutant protein was present but not capable of eliciting locomotor activity.

FIG. 1a represents an exemplary photo of an E18.5 homozygous mutant embryo (marked as Ryr1^{mNisw/Nisw}, on right) in contrast with a heterozygous embryo (Ryr1^{mNisw/+}), where the homozygous mutant demonstrated complete loss of movement, kyphosis (black arrowhead), and carpoptosis (black arrow). FIG. 1b represents exemplary photos of the Ryr1 competent (Ryr1^{+/+}, the left panel) and mutant mouse embryos of Ryr1^{mNisw/Nisw}, Ryr1^{mNisw/tm1Alle} and Ryr1^{tm1Alle/tm1Alle}, where defects such as kyphosis and carpoptosis, are demonstrated. FIG. 1c provides partial nucleic acid and amino acid sequences of the wild type Ryr1 gene (Ryr1) in comparison with the mutated Ryr1 (Ryr1^{mNisw}) gene, where the mutated position in each nucleic acid sequence is marked with an asterisk.

### Example 2

### Mutant RyR1 in Muscle is Non-Functional

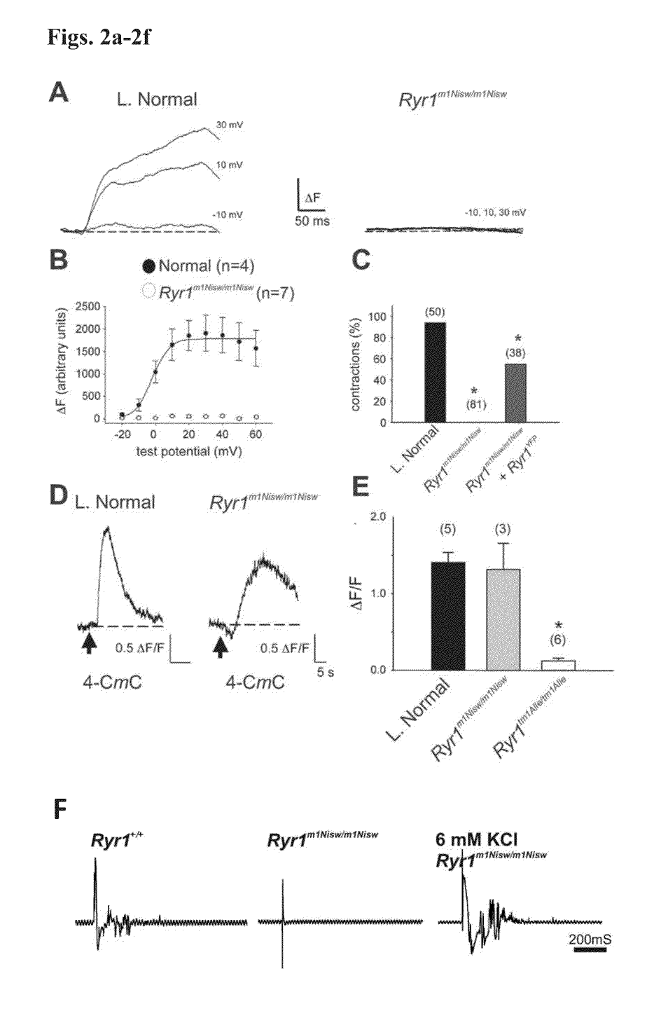

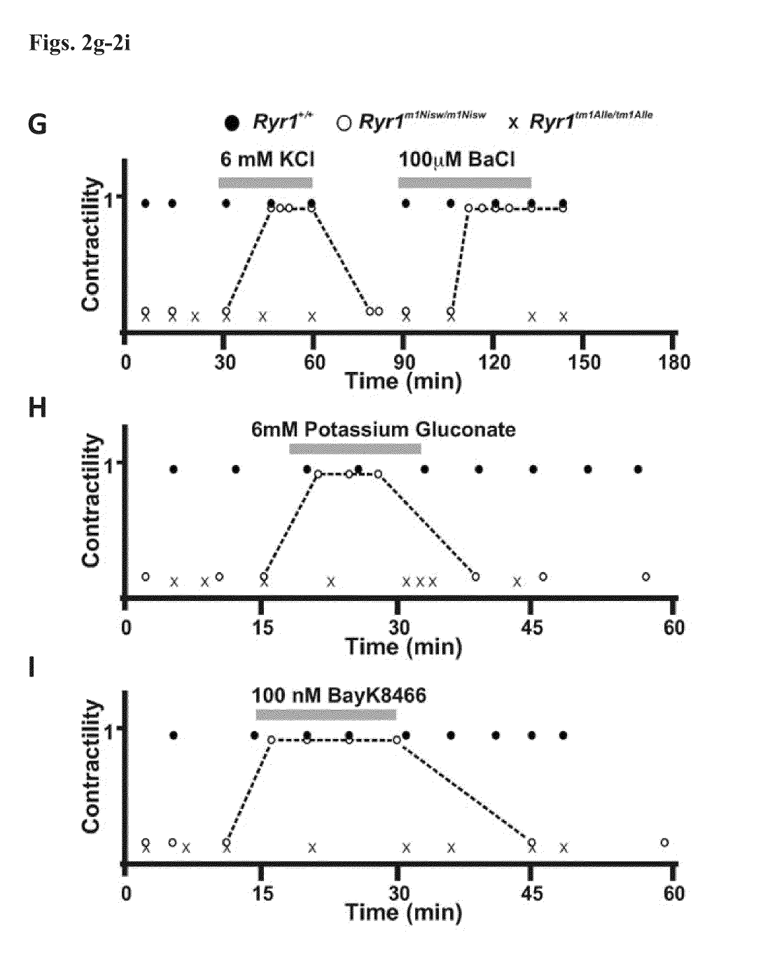

RyR1 functions as a muscle isotype of ryanodine receptors that is mechanically activated by Dihydropyridine receptor (hereinafter “DHPR”) to release Ca²⁺ from the sarcoplasmic reticulum (hereinafter SR), thus evoking excitation-contraction coupling (hereinafter E-C coupling). In some exemplary methods, whole-cell clamping in combination with a fluorescent Ca²⁺ indicator, such as Fluo-3 AM, was used to study the E-C coupling function of RyR1^{m1Nisw} mutant protein in cultured myotubes. Unlike normal myotubes, Ryr1^{m1Nisw/m1Nisw} myotubes failed to release Ca²⁺ from the SR in response to changes in membrane potential (FIGS. 2a-2b). Moreover, Ryr1^{m1Nisw/m1Nisw} myotubes failed to contract in response to electrical stimulation, but this could be rescued by expression of YFP-tagged wild type RyR1 in Ryr1^{m1Nisw/m1Nisw} myotubes (FIG. 2c), indicating that the RyR1 mutation was causative for the non-motile phenotype of homozygous mutant mice. Addition of the RyR1 agonist, 4-chloro-m-cresol (4-CmC; 0.5 mM), produced substantial Ca²⁺ release in both normal and Ryr1^{m1Nisw/m1Nisw} myotubes (peak ΔF/F of 1.4±0.1; n=5 vs. 1.2±0.1; n=3, respectively; p>0.05; FIG. 2d, e), compared to Ryr1^{tm1Alle/tm1Alle} null myotubes (ΔF/F 0.1±0.0; n=6; FIG. 2e). RyR1^{m1Nisw} protein did not lead to depletion of SR Ca²⁺ stores while present in myotubes. However, the rate of SR Ca²⁺ release in response to 4-CmC was much slower in Ryr1^{m1Nisw/m1Nisw} versus normal myotubes (t_{1/2}act=6.5±2.4 vs. 2.2±0.6 s, respectively; p<0.01; FIG. 2d). These results suggested that Ryr1^{m1Nisw} allele encoded a hypomorphic protein that was uncoupled and failed to respond to direct change in membrane potential but were activated by agonists, although the rate of response was significantly impaired. Under controlled physiological conditions, RyR1^{m1Nisw/m1Nisw} protein may be non-functional compared to wild type RyR1 in skeletal muscle.

FIGS. 2a-2e represent exemplary data demonstrating that Ryr1^{m1Nisw/m1Nisw} muscle lacked E-C coupling. FIG. 2a provides representative recordings of myoplasmic Ca²⁺ transients elicited by 50-ms depolarizations from −50 mV to the indicated test potentials for locomotor normal myotubes (left; L. Normal) and Ryr1^{m1Nisw/m1Nisw} myotubes (right). FIG. 2b represents an exemplary graph demonstrating ΔF-V relationships for normal myotubes (•; n=4), and Ryr1^{m1Nisw/m1Nisw} myotubes (∘; n=7). FIG. 2c represents an exemplary histogram illustrating fractions of myotubes that contracted in response to a 100 V, 10 ms electrical stimulus for normal myotubes (black bar), Ryr1^{m1Nisw/m1Nisw} myotubes, and Ryr1^{m1Nisw/m1Nisw} myotubes transfected with YFP-tagged wild type RyR1 (RyR1-YFP, grey bar). The total number of myotubes tested is indicated above each bar. The defect of contraction observed in Ryr1^{m1Nisw/m1Nisw} myotubes (0%) was restored to 50-60% by expression of RyR1-YFP compared to the normal myotubes (90%). FIG. 2d illustrates exemplary diagrams demonstrating changes of Fluo-3 AM fluorescence in response to 4-chloro-m-cresol, a RyR1 agonist (4-CmC, 0.5 mM) elicited in a normal myotube (left) and Ryr1^{m1Nisw/m1Nisw} myotube (right). FIG. 2e represents an exemplary histogram of half-rise time of Ca²⁺ transients in response to 4-CmC observed in normal (blackzbar), Ryr1^{m1Nisw/m1Nisw} (grey bar) and RyR1^{tm1Alle/tm1Alle} (white bar) myotubes. Error bars represent±SEM. An asterisk indicates a significant difference (p=0.006; t-test). The total number of myotubes tested is indicated above each bar.

### Inhibition of K_{IR} Current Induces Muscle Contractions

Muscle from CCD patients shows leakage of Ca²⁺ or uncoupling of RyR1 at the SR. The Ryr1^{m1Nisw} mutation lied outside the channel domain and the 4-CmC agonist experiment indicated that Ca²⁺ stores were not depleted. Therefore, in some exemplary methods, whether voltage dependent orthograde signaling through the DHPR-RyR1 complex could be initiated through changes in membrane potential was first assessed. Within the diaphragm, the resting membrane potential measured via sharp electrode recordings varied between fiber types and in Ryr1^{+/+} muscle this varied between −25 and −70 mV, whereas miniature endplate potentials (mEPPs) only occurred in recordings below −43 mV. Thus, only recordings with mEPPs were quantified. Sharp electrode recordings of Ryr1^{m1Nisw/m1Nisw} and Ryr1^{m1Nisw/+} muscle showed depolarized resting membrane potentials of −48.4+/−3.2 (n=38) and −56.2+/−4.5 (n=26) mV respectively, compared to Ryr1^{+/+} muscle at −61.8+/−4.8 mV (n=42; p<0.01). Membrane potentials of Ryr1^{tm1Alle/tm1Alle} null diaphragms were −53.7+/−4.1 mV (n=21). These data suggested an inability of Ryr1^{m1Nisw} muscle to properly control its resting membrane potential.

In other methods, whether increasing the excitability in E18.5 Ryr1^{m1Nisw/m1Nisw} whole diaphragm muscle could force the DHPR-RyR1 complex to initiate contractility was examined. In one exemplary method, electromyography recordings (EMG) was performed to examine whether whole muscle in vitro was sensitive to potassium-dependent depolarization by adding potassium to the bath to depolarize the membrane. Increasing KCl from 3 mM to 6 mM induced bursts of electrical activity and contractions in Ryr1^{m1Nisw/m1Nisw} muscle but not Ryr1 null muscle (FIGS. 2f-2g). Potassium induced excitability can also be triggered by blocking inward rectifier potassium channels. The addition of BaCl₂, an inhibitor of inward rectifying potassium channels, induced stimulated bursts of activity and contractions in Ryr1^{m1Nisw/m1Nisw} but not Ryr1 null muscle (FIG. 2g) and decreased the resting membrane potential of Ryr1^{m1Nisw/m1Nisw} muscle to −68.5 mV+/−7.1 (n=27). Contractions also transpired when a modified Tyrode's solution supplemented with 6 mM potassium gluconate was applied to the muscle samples, suggesting that the contractions were due to effects on potassium conductance (FIG. 2h). These data provided in vitro evidence that decreasing the resting membrane potential by inhibiting rectifying potassium channels or increasing membrane potential through raising potassium conductance induced contractility of Ryr1^{m1Nisw/m1Nisw} muscle.

DHPR and RyR1 interact through orthograde and retrograde signaling, but the Ryr1^{m1Nisw/m1Nisw} mutation is not located within this interaction region. Orthograde signaling from DHPR opens the RyR1 channel in the SR, while retrograde signaling from RyR1 controls the gating of DHPR. The resumption of contractility after decreasing the resting membrane potential suggests that orthograde signaling between DHPR and RyR1 is intact. To examine orthograde signaling, in one exemplary method, BayK8644 (100 nM), an agonist to the channel portion of DHPR that elicits orthograde signaling, was applied to normal and mutant diaphragm muscles and this induced contractions within minutes in Ryr1^{m1Nisw/m1Nisw} muscle, whereas no contractions occurred in Ryr1^{tm1Alle/tm1Alle} diaphragm muscle (FIG. 2i, the vehicle for BayK8644, ethanol, was used as control). These data indicated that orthograde DHPR-RyR1 signaling was elicited in Ryr1^{m1Nisw/m1Nisw} muscle. Because RyR1-dependent calcium release did not occur under physiological conditions (FIGS. 2a-2b), it suggested that retrograde signaling to allow DHPR gating might be inhibited by RyR1^{m1Nisw/m1Nisw} mutant protein.

E-C coupling in Ryr1^{m1Nisw/m1Nisw} diaphragm muscle was unexpectedly found to be reactivated through pharmacological agents and increased extracellular K⁺ considering myotube culture experiments which indicated that this channel was non-functional. This finding emphasized the importance of studies in intact muscle to determine RyR1 functionality. The mutation was localized in a region of RyR1 that was important for binding to DHPR. Direct DHPR-RyR1 interactions may be necessary for both orthograde and retrograde communication. BayK8644 induced E-C coupling in Ryr1^{m1Nisw/m1Nisw} muscle (FIG. 2i), suggesting that orthograde signaling could be initiated but that retrograde signaling could be affected, perhaps due to defects in DHPR gating by RyR1. Binding studies of DHPR-RyR1 suggested that RyR1 conformational changes were necessary for skeletal muscle E-C coupling. The receptor region of RyR1 has several phosphorylation sites and also contains binding sites for protein modulators and second messengers that modulate E-C coupling, such as ATP.

FIGS. 2f-2i represent exemplary data demonstrating that Ryr1^{m1Nisw/m1Nisw} muscle regained contractility given increased potassium. FIG. 2f provides exemplary diagrams of electromyography recordings (EMG) of diaphragm muscle from Ryr1^{+/+}, Ryr1^{m1Nisw/m1Nisw}, or Ryr1^{m1Nisw/m1Nisw} treated with 6 mM KCl, where the treatment of KCL improved electrical activity produced by Ryr1^{m1Nisw/m1Nisw} muscles. FIG. 2g represents an exemplary graph of contractility of E18.5 diaphragm muscle from Ryr1^{+/+}, Ryr1^{m1Nisw/m1Nisw}, or RyR1^{tm1Alle/tm1Alle} embryos. After equilibration, all samples were bathed first in 6 mM KCl, followed by wash out and then addition of 100 μm BaCl as indicated. Compared to normal contraction, Ryr1 null muscle failed to contract in response to either KCl or BaCl stimulation while contraction in Ryr1^{m1Nisw/m1Nisw} muscle was rescued by both (course of three separate experiments showing either contraction (value=1) or no contraction (value=0)). FIG. 2h provides an exemplary graph of contractility of E18.5 diaphragm muscle from Ryr1^{+/+}, Ryr1^{m1Nisw/m1Nisw} and RyR1^{tm1Alle/tm1Alle} embryos in 6 mM potassium gluconate (n=4). FIG. 2i represents an exemplary graph demonstrating contractility of E18.5 diaphragm muscle from Ryr1^{m1Nisw/m1Nisw} and RyR1^{tm1Alle/tm1Alle} embryos following addition of 100 nM BayK8644 (n=4).

### Example 3

### Ryr1^{m1Nisw/+} Heterozygotes Show Clinical and Pathological Symptoms of CCD

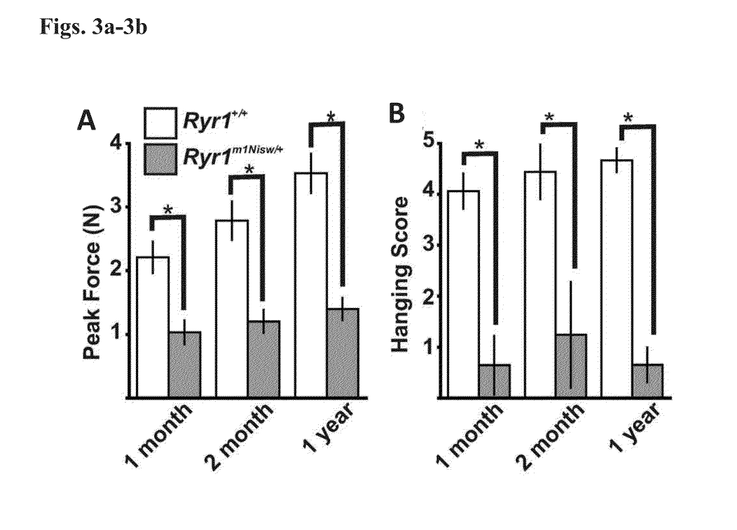

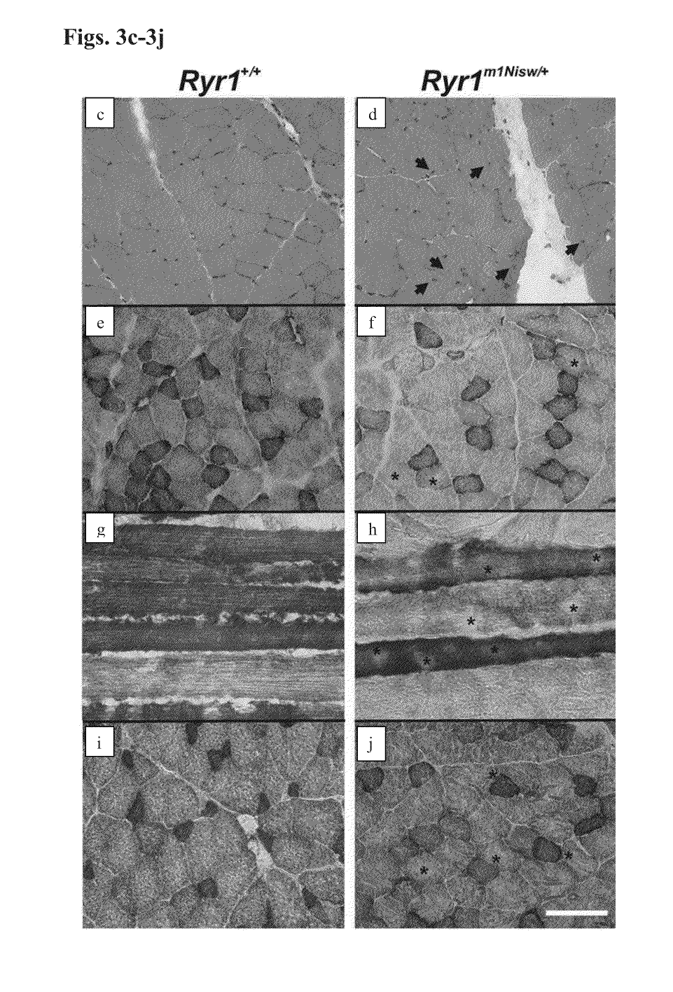

In other exemplary methods, muscle deficits in heterozygous mice were studied. For example, muscle weakness was quantified by grip strength tests and wire hanging task. Ryr1^{m1Nisw/+} mice did not display muscle wasting but they did show muscle weakness by these tests. One-month, two-month and one-year old RyR1^{m1Nisw/+} and Ryr1^{+/+} mice were tested on a control diet (0.6% potassium). RyR1^{m1Nisw/+} mice showed a statistically significant decrease in grip strength (measured in Newtons, FIG. 3a) and hanging task (FIG. 3b, see Materials and methods) when compared to age matched Ryr1^{+/+} mice. Thus, mice that were heterozygous for the mutation showed muscle weakness, the most poignant clinical manifestation of CCD. CCD patients with mutations in RyR1 can be at risk for malignant hyperthermia (MH). However, Ryr1^{m1Nisw/+} heterozygous mice did not show evidence of MH (experiments performed on 2-month-old animals, data not shown).

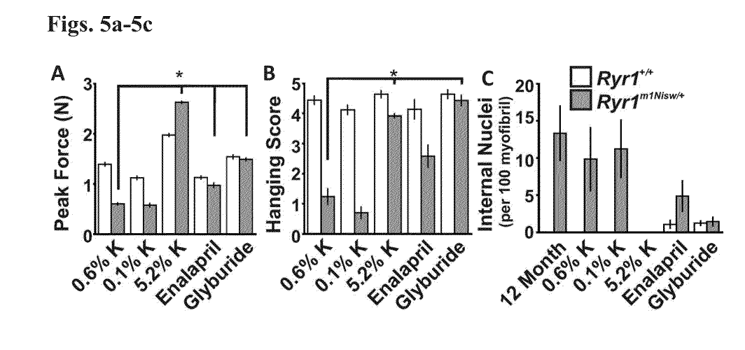

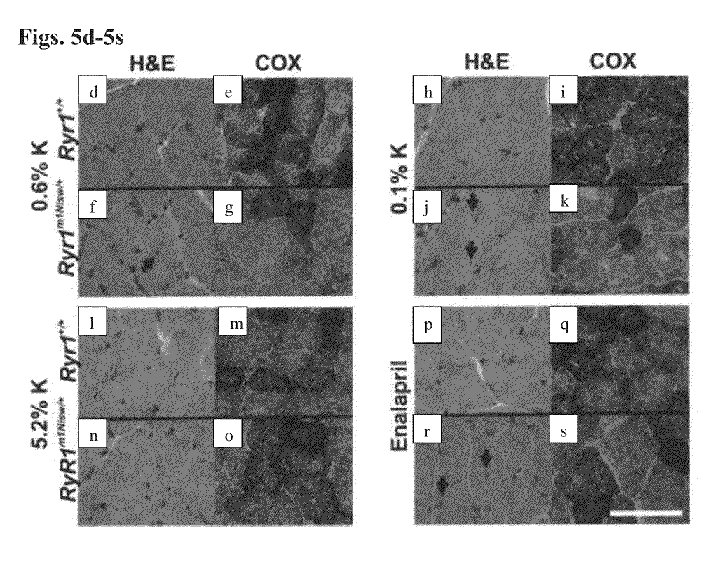

CCD is diagnosed and subclassified by histopathological analysis using stains such as cytochrome oxidase (COX) and nicotinamide adenine dinucleotide hydride-tetrazolium reductase (NADH-TR) which highlight mitochondria and ATP synthesis, and hematoxylin and eosin stain (H&E) to mark nuclei. In normal muscle, nuclei are located at the periphery of the myofiber and the mitochondria reside throughout the myofiber. Muscle biopsies of CCD patients show centrally located nuclei, as well as disorganized areas in the center of the myofiber called cores that lack mitochondria and are devoid of metabolic activity; these occur in some but not all muscles and are reflective of cellular damage. In one exemplary method, 2-month and 1-year old RyR1^{m1Nisw/+} vastus lateralis muscle was analyzed to demonstrate centrally located nuclei, central cores and decreased COX staining relative to wildtype Ryr1^{+/+} muscle (1 yr data in FIGS. 3c-3j and 2 month data in FIGS. 5c-5g). The muscle weakness and histopathology indicated that this novel RyR1^{m1Nisw} allele represented an excellent disease model of CCD.

FIG. 3 provides exemplary data demonstrating that Ryr1^{m1Nisw/+} pathology mimics clinical CCD. FIG. 3a presents an exemplary histogram of average grip strength assayed from Ryr1^{+/+} and Ryr1^{m1Nisw/+} mice using vertical digital push-pull strain gauge at different ages (1-month-old, 2-month-old and 1-year-old). Defects were observed in each age group of Ryr1^{m1Nisw/+} mice compared to normal mice. FIG. 3b represents an exemplary histogram demonstrating hanging task determination of upper body strength of Ryr1^{+/+} and Ryr1^{m1Nisw/+} mice at different ages (10 trials/mouse, n=5 per set). Ryr1^{m1Nisw/+} mice demonstrated significant upper body muscle weakness at each age group compared to normal mice. FIGS. 3c-3d represent exemplary H&E staining results indicating central nuclei (arrows) in Ryr1^{m1Nisw/+} (d) but not Ryr1^{+/+} (c) vastus lateralis muscle from one year old mice. FIGS. 3e-3h present exemplary NADH-TR staining results indicating cores in femoral muscle of Ryr1^{m1Nisw/+} (indicated by asterisks) but not Ryr1^{+/+}. FIGS. 3i-3j provide exemplary cytochrome oxidase (COX) staining images of mitochondria denote a decrease (asterisks) in mitochondria in femoral muscle of Ryr1^{m1Nisw/+} (j) but not Ryr1^{+/+} (i). Scale bar=50 μm in FIGS. 3c-3j.

### Example 4

### Ryr1^{m1Nisw/m1Nisw} Muscle have Decreased ERK2 Protein and Na⁺, K⁺ ATPase Phosphorylation and Increased Resting Membrane Potential

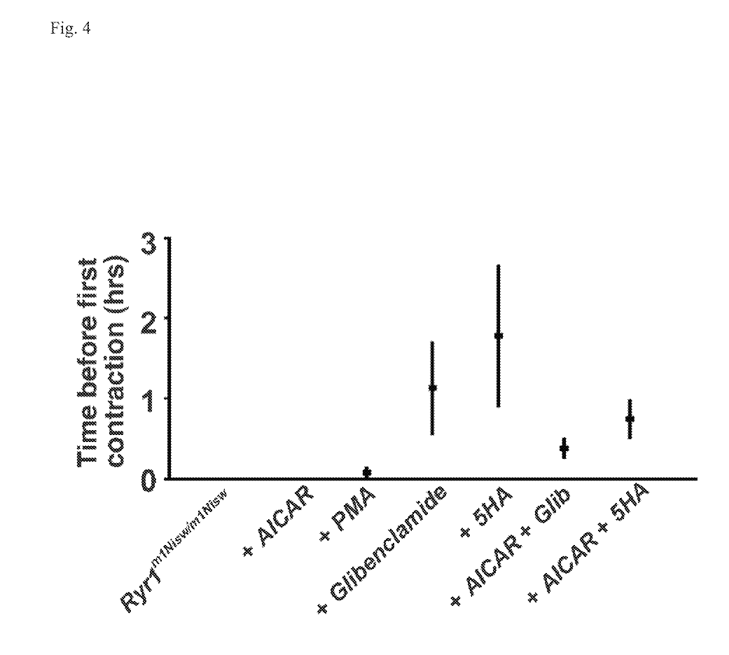

The ability of an increase in potassium levels to elicit contractility in the mutant muscle led to studies of whether there was an alteration in potassium transport across the membrane in Ryr1^{m1Nisw/m1Nisw} mutants. In myotubes, the application of ryanodine at levels that activate RyR increases ERK1/2 protein levels and c-Fos and c-Jun transcript levels. ERK1/2 phosphorylates the most abundant potassium transport protein, Na⁺, K⁺-ATPase, which, in turn, induces an increase in ion transport. Na⁺, K⁺-ATPase is essential for maintaining the equilibrium potential of Na⁺ and K⁺ at the membrane. In one exemplary method, western blot was performed to analyze certain protein levels in Ryr1^{m1Nisw/m1Nisw} and Ryr1^{tm1Alle/tm1Alle} embryonic muscle extracts with beta-tubulin served as a loading control. ERK2 protein levels were found to be significantly reduced in the mutants compared to Ryr1^{+/+} littermate muscle extracts (data not shown). The normalized protein levels of the pump subunits, Na⁺, K⁺-ATPase α1 and α2 were increased in Ryr1^{m1Nisw/m1Nisw} and Ryr1^{m1Nisw/+}. The ERK1/2 dependent phosphorylation of Ser-23 on Na⁺, K⁺-ATPase α1 was decreased in Ryr1^{m1Nisw/m1Nisw} and Ryr1^{m1Nisw/+}. In Ryr1^{tm1Alle/tm1Alle} muscle extracts, the ratio was increased compared to Ryr1^{+/+} muscle littermate extracts (data not shown). This data was consistent with the observed decrease in ERK2 protein levels (data not shown). In another exemplary method, Phorbol-12-myristate-13-acetate (PMA 50 nM), an activator of ERK1/2 phosphorylation, was applied to the bath of in vitro Ryr1^{m1Nisw/m1Nisw} diaphragm preparation to increase the phosphorylation of Na⁺, K⁺-ATPase. Within 5 minutes of PMA addition, Ryr1^{m1Nisw/m1Nisw} myofibers contracted after electrical stimuli and continued until 30-minute washout (FIG. 4). Application of PMA decreased the membrane potential −18.3+/−5.3 mV to −67.7 mV (Ryr1^{m1Nisw/m1Nisw} mutant in ethanol at −49.4+/−5.6 n=14). PMA addition had no effect on Ryr1^{tm1Alle/tm1Alle} null diaphragm muscle. These data suggested that the Na⁺, K⁺-ATPase did not function properly in Ryr1^{m1Nisw/m1Nisw} mutants and hence the membrane was more depolarized. This might occur as a consequence of reduced ERK1/2 mediated phosphorylation of Na⁺, K⁺-ATPase. These data suggested that molecular pathways required for transport of serum potassium between the muscle and the interstitium were impaired in the presence of the mutant RyR1^{m1Nisw} protein.

### Example 5

### Molecular Pathways Involved in Membrane Repolarization are Altered in Ryr1^{m1Nisw/m1Nisw} Muscle

In other methods repolarization of membranes were examined. ERK1/2 acts as a potent regulator of transcription. Therefore, the RNA expression of several transcripts involved in potassium homeostasis was examined by real time PCR using E18.5 limb muscles from Ryr1^{+/+}, Ryr1^{m1Nisw/+}, Ryr1^{m1Nisw/m1Nisw}, and Ryr1^{tm1Alle/m1Nisw} embryos (Table 1). No significant changes were observed in the expression of Scn4a which encodes the sodium channel required for the propagation of action potentials in t-tubules, or Kcna1 encoding the potassium voltage-gated channel. However, there was decreased expression of Kcnj2 and Kcnj8, the genes encoding the strong inward rectifying channel K_{IR}2.1 and the K_{ATP}6.1 channel, respectively, in Ryr1^{m1Nisw/m1Nisw} and Ryr1^{tm1Alle/tm1Alle} muscle. These levels, however, were unchanged in Ryr1^{m1Nisw/+} compared to Ryr1^{+/+} muscle. The t-tubule and plasma membrane K_{ATP}6.2 channel (Kcnj11) was downregulated in Ryr1^{m1Nisw/m1Nisw} muscle but not significantly changed in Ryr1^{m1Nisw/+} or Ryr1^{tm1Alle/tm1Alle} muscle. These data suggest that the expression of potassium channels involved in control of the resting membrane potential are downregulated in Ryr1^{m1Nisw/m1Nisw} muscle.

K_{ATP} channels actuate E-C coupling in fast-twitch muscle fibers. In certain exemplary methods, expression profiles of modulators of these channels were examined. Muscle samples were isolated from E18.5 embryos derived from mothers fed control 0.6% K diet. The sulfonylurea receptors (SUR) are ATP-binding cassette (ABC) transporters that bind to K_{ATP} channels. Like the K_{ATP} channels, SUR2 (abcc9), was downregulated in Ryr1^{m1Nisw/m1Nisw} and Ryr1^{tm1Alle/tm1Alle} muscle but not in Ryr1^{m1Nisw/+} compared to Ryr1^{+/+} muscle (Table 1). However, SUR1 (abcc8) was significantly upregulated in Ryr1^{m1Nisw/+}, Ryr1^{m1Nisw/m1Nisw}, and Ryr1^{tm1Alle/tm1Alle} muscle. Conversely, Il15Ra, which is involved in fatigue resistance, exercise capacity, and muscle subtype specification, showed no significant difference between all Ryr1 alleles.

These results indicated the downregulation of a number of genes that mediate potassium transport across the membrane in Ryr1^{m1Nisw/m1Nisw} limb muscle. Because changes in mRNA levels are not always reflected by similar changes in protein expression levels, in other exemplary methods, protein levels were examined from Ryr1^{m1Nisw/+}, Ryr1^{m1Nisw/m1Nisw} and Ryr1^{tm1Alle/tm1Alle} E18.5 embryonic muscle and compared against littermate Ryr1^{+/+} muscle. Ryr1^{tm1Alle/tm1Alle} muscle consistently showed decreased expression of membrane potassium channels. Ryr1^{m1Nisw/m1Nisw} muscle showed a similar pattern of protein expression with the exception of KIR2.1, which showed elevated protein levels (data not shown). These data suggested that the expression of KIR2.1 and KATP channels was influenced by the lack of internal release of calcium stores. Furthermore, lack of rectification of the resting membrane potential after an action potential, and the lack of excitability of the mutant muscle might be due to altered expression of these channels in mice with uncoupled RyR1^{m1Nisw/m1Nisw} protein.

TABLE 1
Quantitative RT-PCR of E18.5 Limb Muscle

| Gene | Protein | Ryr1^{+/+} | Ryr1^{m1Nisw/+} | Ryr1^{m1Nisw/m1Nisw} | Ryr1^{tm1Alle/tm1Alle} |
|---|---|---|---|---|---|
| Kcnj2 | K_{IR}2.1 | 1.00 +/− 0.11 | 1.11 +/− 0.24 | 0.70 +/− 0.19*** | 0.76 +/− 0.06* |
| Kcnj8 | K_{ATP}6.1 | 1.00 +/− 0.09 | 1.08 +/− 0.20 | 0.69 +/− 0.06*** | 0.74 +/− 0.08*** |
| Kcnj11 | K_{ATP}6.2 | 1.00 +/− 0.15 | 0.93 +/− 0.12 | 0.59 +/− 0.15*** | 1.02 +/− 0.32 |
| Scn4a | Na_{V}1.4 | 1.00 +/− 0.21 | 1.04 +/− 0.23 | 0.86 +/− 0.07 | 1.19 +/− 0.08 |
| Prkaa1 | AMPK | 1.00 +/− 0.14 | 0.68 +/− 0.19*** | 0.72 +/− 0.14*** | 0.74 +/− 0.07*** |
| Abcc8 | SUR1 | 1.00 +/− 0.06 | 1.66 +/− 0.24*** | 1.97 +/− 0.42*** | 2.36 +/− 0.46*** |
| Abcc9 | SUR2 | 1.00 +/− 0.19 | 0.99 +/− 0.46 | 0.72 +/− 0.11* | 0.65 +/− 0.16* |
| Il15Ra | IL15Ra | 1.00 +/− 0.27 | 1.02 +/− 0.19 | 0.97 +/− 0.24 | 1.16 +/− 0.38 |
| Atp1a1 | N⁺,K⁺ATPaseα1 | 1.00 +/− 0.19 | 1.03 +/− 0.14 | 1.11 +/− 0.31 | 1.27 +/− 0.22 |
| Atp1a2 | N⁺,K⁺ATPaseα2 | 1.00 +/− 0.27 | 1.52 +/− 0.24 | 1.07 +/− 0.40 | 1.40 +/− 0.39 |
| Kcna1 | Kᵥ1.1 | 1.00 +/− 0.22 | 0.98 +/− 0.24 | 0.97 +/− 0.06 | 1.36 +/− 0.17 |

Values normalized to Tbp before normalization to Ryr1^{+/+} control.
Asterisks indicates significant value *= 0.05, **= 0.001, ***= 0.005.

Stimulation of AMPK Activity can Restore the Membrane Potential in Ryr1m1Nisw/m1Nisw Muscle

Potassium transport activity of K_{IR} and K_{ATP} channels is regulated by the upstream AMP-activated protein kinase (AMPK), which phosphorylates and inhibits K_{IR}2.1 while assisting the activation of K_{ATP}6.1, and K_{ATP}6.2 channel functions. The catalytic transcript of AMPK (encoded by Prkaa1) showed significant downregulation in Ryr1^{m1Nisw/+}, Ryr1^{m1Nisw/m1Nisw}, and Ryr1^{tm1Alle/tm1Alle} E18.5 muscle (Table 1). AMPK protein levels were also reduced in Ryr1^{m1Nisw/m1Nisw} and Ryr1^{tm1Alle/tm1Alle} but Ryr1^{m1Nisw/+} not Ryr1^{m1Nisw/+} embryonic muscle extracts (data not shown).

In certain exemplary methods, restoration of membrane potential and muscle contractions by pharmacological stimulation was tested. First, effect of stimulation of AMPK activity on restoring the resting membrane potential of the mutant diaphragm muscle was determined. An AMPK activator, e.g. 5-aminoimidazole-4-carboxamide-1-β-d-ribofuranoside (AICAR, 500 μM), was bath applied to explanted Ryr1^{m1Nisw/m1Nisw} E18.5 diaphragm muscles. AICAR was unable to initiate contractions during electrical stimulation of Ryr1m1Nisw/m1Nisw and Ryr1tm1Alle/tm1Alle muscle. However, sharp electrode recordings of Ryr1^{m1Nisw/m1Nisw} diaphragm muscle with AICAR showed a −10.4+/−3.9 mV (n=18) shift in membrane potential, indicating that increased activation of AMPK can restore the mutant resting membrane potential to normal levels (FIG. 4, Table 2 and data not shown). Next, other pharmacological stimuli were examined. SUR1 (abcc8) and SUR2 (abcc9) also inhibit the activities of K_{ATP} channels. Glibenclamide (2 μM) and 5-hydrodecanoic acid (5HA, 200 μM) selectively bind to SUR1 and SUR2 to block ATP-sensitive K⁺ channels. Glibenclamide treatment normalized the resting membrane potential and both glibenclamide and 5HA elicited contractions in Ryr1^{m1Nisw/m1Nisw} diaphragms after 2-hour bath application (n=6 for each) with 50% of the explants showing contractions after 1 hour (FIG. 4, Table 2 and data not shown). The combination of glibenclamide and AICAR elicited contraction within 10 minutes of bath application in all Ryr1^{m1Nisw/m1Nisw} diaphragm muscles (n=4), as did the combination of 5HA and AICAR. Pharmacological activation of ERK1/2 with Phorbol-12-myristate-13-acetate (PMA) normalized the membrane potential and strongly elicited contractions (FIG. 4, Table 2 and data not shown). Thus, increasing extracellular potassium, directly altering potassium-coupled transport mechanisms or activating internal calcium release could restore RyR1-dependent muscle contractions. Moreover these data suggested that functional RyR1 protein was required for molecular pathways regulating potassium transport that control membrane potential in muscle and that pharmacological restoration of these activities was sufficient to restore E-C coupling in Ryr1^{m1Nisw/m1Nisw} diaphragms.

TABLE 2
EMG recordings of Ryr1^{m1Nisw/m1Nisw} E18.5 diaphragms
in the presence of certain pharmacological stimuli

| | | Affected | Minutes to |
|---|---|---|---|
| | | K⁺ Transport | Contraction |
| Stimulation | Action | Protein | (Stand. Dev) |
| None | Control Solution | N/A | None |
| AICAR | AMPK activator | K_{IR}2.1, K_{ATP}6.x | None |
| Glib | Inhibit SUR1 & SUR2 | K_{ATP}6.x | 67 (34) |
| 5HA | Inhibit SUR1 & SUR2 | K_{ATP}6.x | 106 (52) |
| AICAR + 5HA | See Above | K_{IR}2.1, K_{ATP}6.x | 22 (7) |
| AICAR + Glib | See Above | K_{IR}2.1, K_{ATP}6.x | 44 (13) |
| PMA | ERK activator | Na⁺, K⁺ ATPase | 4 (2) |

Activation of ERK1/2 and AMPK via pathway agonists was sufficient to reestablish the resting membrane potential in Ryr1^{m1Nisw/m1Nisw} muscle. While ERK1/2 agonist could induce muscle contractions in Ryr1^{m1Nisw/m1Nisw} muscle within minutes, increased phosphorylation of AMPK could not induce contractions unless combined with the inhibition of the K_{ATP} channel-specific regulator sulfonylurea receptor (SUR). These data suggested that the open probability (P_{O}) of K_{ATP} channels was increased in Ryr1^{m1Nisw/m1Nisw} muscle upon activation of AMPK and inhibition of SUR. However, AMPK also inhibited the P_{O} of K_{IR} channels. BaCl₂, which can inhibit K_{IR} channels, and PMA, which increases Na⁺, K⁺ ATPase activation, both decrease resting membrane potential in Ryr1^{m1Nisw/m1Nisw} muscle, as did increasing the concentration of extracellular K⁺. As a result, Na_{V}1.4 channels returned to an active state and the ability to elicit an action potential was restored. These data suggest that functional RyR1 is indirectly affecting potassium transport across the membrane. However, potassium-induced depolarization could rescue RyR1^{m1Nisw} protein function.

Embryonic Ryr1^{m1Nisw/m1Nisw} muscle had decreased ERK2 and AMPK expression and decreased phosphorylation of these proteins, which in turn could affect membrane potential and contractility. While these proteins have not been shown to directly interact with RyR1, disruption in their expression and activity can perturb the mitochondrial production of ATP through downstream mechanisms. In addition, KATP channel transcript and protein were downregulated in embryonic Ryr1^{m1Nisw/+} muscle, and Ryr1^{m1Nisw/+} adult muscle showed a decrease in mitochondrial number, both of which could inhibit ATP production. One possibility could be that these modulators may affect retrograde signaling between RyR1 and DHPR. These data suggests that the RyR1^{m1Nisw} protein inhibit the activation state of DHPR, resulting in decreased KATP channel-dependent mitochondrial function and kinase activation. Conformational changes in the receptor region of RyR1 may act as a ratchet during states of orthograde and retrograde signaling. Additionally, a feedback relationship between internal calcium dynamics and membrane potential might be required for proper E-C coupling.

FIG. 4 provides an exemplary graph demonstrating contractility of electrically stimulated diaphragm muscle from RyR1^{m1Nisw/m1Nisw} E18.5 embryos alone or in the presence of PMA (50 nM), AICAR (500 μM), Glibenclamide (2 μM), 5-hydrodecanoic acid (5HA, 200 μM), AICAR plus Glibenclamide, or AICAR plus 5 HA.

### Example 6

### Potassium Supplementation can Rescue the Clinical, Pathological and Potential Molecular Features of CCD in Ryr1^{m1Nisw/+} Heterozygous Mice

In one exemplary method, whether the manifestations of CCD in vivo could be altered by changes in serum potassium in the Ryr1^{m1Nisw/+} heterozygous mice were investigated. In mice, in vivo serum potassium can be modulated through diet or through angiotensin-converting enzyme (ACE) inhibitors, which increases serum potassium levels. In other methods, the effect of altered serum potassium levels on in vivo muscle function was examined. Ryr1^{m1Nisw/+} and Ryr1^{+/+} mice were raised on 0.6% K (the level of potassium in standard mouse chow) for four weeks (muscle weakness was already observed at this age, FIG. 3) and then fed either a 0.1% or 5.2% potassium diet to provide hypokalemic or hyperkalemic conditions, respectively. Alternatively, the mice were given an ACE inhibitor, such as Enalapril (0.12 g/l added to the water supply on 0.6% K diet) for an additional four weeks, or Glyburide (15 mg/kg/day oral gavage) for a month. Weakness in Ryr1^{m1Nisw/+} mice was clearly evident on 0.1% K and 0.6% K diets with an up to 50% reduction in grip strength and an inability to hang onto the wire (FIGS. 5a-5b). Most dramatically, on 5.2% K diets, grip strength was fully rescued and even increased in RyR1^{m1Nisw/+} mice compared to Ryr1^{+/+} mice (FIG. 5a). Muscle strength, as assayed in the hanging task, was also rescued, although RyR1^{+/+} mice often escaped, while RyR1^{m1Nisw/+} mice stayed on the wire with all four paws until the test was completed (FIG. 5b). Ryr1^{m1Nisw/+} mice administered Enalapril or Glyburide showed increased grip strength and muscle strength compared to RyR1^{m1Nisw/+} on control diets. These data demonstrated that potassium supplementation rescued muscle weakness caused by the RyR1^{m1Nisw} mutation.

Pathological examination is the hallmark diagnosis of CCD. In one exemplary method, the effects of diet on muscle histology were studied. Centrally located nuclei and decreased mitochondrial activity were detected in Ryr1^{m1Nisw/+} vastus lateralis muscle compared to Ryr1^{+/+} muscle in low potassium and control diets (FIGS. 5c-5k). Strikingly, Ryr1^{m1Nisw/+} muscle on high potassium diet resembled RyR1^{+/+} muscle in that it lacked centrally-located nuclei and showed a rescue of mitochondrial activity (FIGS. 5c and 5l-5o). Enalapril, demonstrated partial rescue of muscle strength, was applied and demonstrated a mild improvement in muscle histology (FIGS. 5p-5s). Glyburide treated samples demonstrated lack of centrally-located nuclei (5c). These data at least suggested that the pathology of CCD could be rescued with increased extracellular serum potassium or treatment with Enalapril or Glyburide even after observable muscle dysfunction, providing evidence for a potential treatment for CCD through diet or drugs already used for other clinical treatments. In addition, the CCD phenotype was also rescued by 5-hydrodecanoic acid (5HA), which blocks ATP-sensitive K⁺ channels (FIG. 4 and Table 2). Moreover, these data demonstrate that there is a significant correlation between muscle strength and muscle pathology.

Some diseases associated with RyR1 mutations, such as MH, are sensitive to potassium levels therefore; a testing method in patients or biopsy samples would likely be needed prior to providing the patient with potassium supplements.

FIG. 5 represents exemplary data demonstrating rescue of muscle strength and presenting histology of Ryr1^{m1Nisw/+} muscle on increased potassium diet demonstrating reversal of CCD pathology. FIG. 5a represents an exemplary histogram comparing average grip strength observed from 2 month old Ryr1^{+/+} (white bar) and Ryr1^{m1Nisw/+} (grey bar) mice using vertical digital push-pull strain gauge on the different diets, Enalapril and Glyburide (15 mg/kg/day oral gavage) (five trials/mouse and 5 mice/set; p values<0.001). FIG. 5b provides an exemplary histogram demonstrating in vivo hanging task determination of upper body strength of Ryr1^{+/+} and Ryr1^{m1Nisw/+} mice for all conditions (10 trials/mouse, n=5 per set; p values<0.001). FIG. 5c represents an exemplary histogram of quantified number of central nuclei per 100 myofiber from vastus lateralis muscles from Ryr1^{+/+} and Ryr1^{m1Nisw/+} mice on the various potassium diets, Enalapril and Glyburide (n=10 per muscle, n=3 per set of muscles for total of n=30; p values<0.001, error bar in SEM). FIGS. 5d-5s present exemplary cross-section staining images of vastus lateralis myofiber from Ryr1^{+/+} and Ryr1^{m1Nisw/+} mice on the various potassium diets and Enalapril stained with H&E (left panels) and COX (right panels). Ryr1^{m1Nisw/+} mice on 5.2% K diet showed increased COX staining and no central nuclei, similar to Ryr1^{+/+}. These pathological features were still observed in Ryr1^{m1Nisw/+} mice on 0.1% and 0.6% K diets. Scale bar=50 μm.

### Example 7

### Exemplary Biomarkers

In some exemplary methods, biomarkers of CCD and disease recovery were defined. Genes, such as molecular markers of potassium homeostasis that were found to be disrupted in E18.5 embryos were studied. Quantitative RT-PCR was conducted on three separate muscles: vastus lateralis, tibialis anterior and adductor magnus isolated from 2-month-old wild type and heterozygous Ryr1^{m1Nisw/+} mice that had been kept on low potassium, control or high potassium diets for 4 weeks. Kcnj8 was downregulated in Ryr1^{m1Nisw/+} adult muscle in low potassium and control diets but was restored to normal levels in high potassium diet (the latter compared to RyR1^{+/+} mice on control or high diets, Table 3). Moreover, Prkaa1 was significantly upregulated in Ryr1^{m1Nisw/+} muscle in low potassium and control diets and in low potassium muscle from RyR1^{+/+} mice but returned to normal levels in Ryr1^{m1Nisw/+} on high potassium diet. Abcc8 transcripts increased in all potassium diets, while Na⁺, K⁺-ATPase α1 transcripts were increased in low and control potassium diet, but were considerably decreased in high potassium diet. Na⁺, K⁺-ATPase α2 protein decreased in Ryr1^{m1Nisw/+} muscle on low potassium and control diets and was restored to normal levels on high potassium diet (Table 3).

TABLE 3
Quantitative RT-PCR of muscles from wild type and heterozygous RyR1^{m1Nisw/+} mice

| | 0.6% K Diet | | 0.1% K Diet | | 5.2% K Diet | |
|---|---|---|---|---|---|---|
| Gene | Ryr1^{+/+} | Ryr1^{m1Nisw/+} | Ryr1^{+/+} | Ryr1^{m1Nisw/+} | Ryr1^{+/+} | Ryr1^{m1Nisw/+} |
| Kcnj8 | | | | | | |
| Vastus lateralis | 1.00 +/− 0.20 | 0.56 +/− 0.12*** | 0.89 +/− 0.04 | 0.53 +/− 0.05*** | 0.97 +/− 0.20 | 0.97 +/− 0.23 |
| Tibialis anterior | 1.00 +/− 0.13 | 0.74 +/− 0.19*** | 1.15 +/− 0.20 | 0.72 +/− 0.06*** | 0.87 +/− 0.11 | 0.91 +/− 0.12 |
| Adductor magnus | 1.00 +/− 0.24 | 0.69 +/− 0.16** | 1.11 +/− 0.16 | 0.69 +/− 0.05*** | 1.16 +/− 0.20 | 1.08 +/− 0.17 |
| Prkaa1 | | | | | | |
| Vastus lateralis | 1.00 +/− 0.14 | 1.23 +/− 0.15** | 1.20 +/− 0.05*** | 1.35 +/− 0.08*** | 1.11 +/− 0.10 | 1.07 +/− 0.14 |
| Tibialis anterior | 1.00 +/− 0.04 | 1.11 +/− 0.06*** | 1.13 +/− 0.02*** | 1.21 +/− 0.05*** | 1.08 +/− 0.05 | 1.07 +/− 0.10 |
| Adductor magnus | 1.00 +/− 0.11 | 1.20 +/− 0.11** | 1.27 +/− 0.08*** | 1.31 +/− 0.10*** | 1.01 +/− 0.11 | 1.10 +/− 0.06 |
| Abcc8 | | | | | | |
| Vastus lateralis | 1.00 +/− 0.07 | 1.30 +/− 0.28*** | 0.68 +/− 0.10*** | 1.26 +/− 0.15** | 1.05 +/− 0.30 | 1.51 +/− 0.33* |
| Tibialis anterior | 1.00 +/− 0.04 | 1.20 +/− 0.17*** | 0.67 +/− 0.05*** | 1.23 +/− 0.14*** | 1.09 +/− 0.23 | 1.31 +/− 0.27* |
| Adductor magnus | 1.00 +/− 0.12 | 1.28 +/− 0.22* | 0.74 +/− 0.08*** | 1.43 +/− 0.24*** | 0.98 +/− 0.23 | 1.61 +/− 0.42* |
| Atpa1 | | | | | | |
| Vastus lateralis | 1.00 +/− 0.15 | 1.23 +/− 0.21* | 1.04 +/− 0.10 | 1.48 +/− 0.31** | 0.94 +/− 0.66 | 0.18 +/− 0.06*** |
| Tibialis anterior | 1.00 +/− 0.21 | 1.22 +/− 0.13* | 1.09 +/− 0.07 | 1.34 +/− 0.29* | 0.75 +/− 0.70 | 0.23 +/− 0.06*** |
| Adductor magnus | 1.00 +/− 0.26 | 1.25 +/− 0.17* | 1.07 +/− 0.18 | 1.30 +/− 0.22* | 0.93 +/− 0.55 | 0.24 +/− 0.07*** |

Values normalized to Gapdh before normalization to Ryr1^{+/+} control on 0.6% diet.
Asterisks indicates significant value
*= 0.05,
**= 0.001,
***= 0.005.

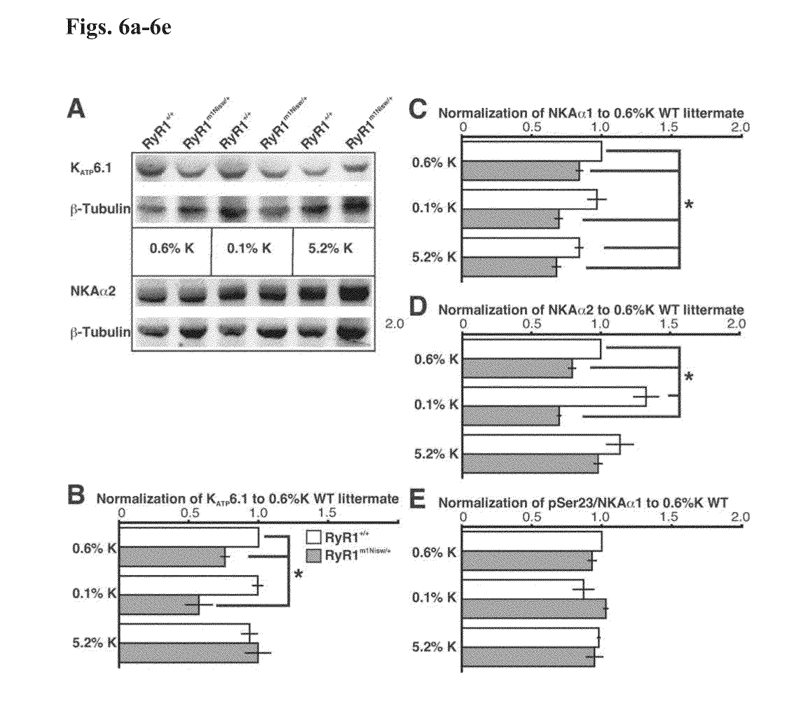

The pathology of Ryr1^{m1Nisw/+} muscle showed that mitochondrial activity was influenced by potassium diet. Because K_{ATP}6.1 channels (encoded by Kcnj8) are located in mitochondria, K_{ATP}6.1 channel expression should be decreased in Ryr1^{m1Nisw/+} muscle in the low and control diets. To test this, in one exemplary method, western blot was performed to examin vastus lateralis muscles in Ryr1^{m1Nisw/+} and Ryr1^{+/+} mice after one-month exposure to the various diets. K_{ATP}6.1 expression was decreased in Ryr1^{m1Nisw/+} adult muscle in low potassium and control diets (all normalized to RyR1^{+/+} mice on control potassium diet, FIGS. 6a-6b). Remarkably, in Ryr1^{m1Nisw/+} mice on high potassium diet, K_{ATP}6.1 expression was restored to RyR1^{+/+} muscle protein levels (FIGS. 6a-6b). Hence, K_{ATP}6.1 expression might be a marker for decreased mitochondrial activity in CCD.

K_{ATP}6.1 channels are also present in the cell membrane, as are Na⁺, K⁺-ATPases. Therefore, in another exemplary method, Na⁺, K⁺-ATPase protein expression and phosphorylation of Na⁺, K⁺-ATPase α1 on Serine 23 were examined. The ratio of pSer23 to Na⁺, K⁺-ATPase α1 was unchanged compared to Ryr1^{+/+} muscle, however Na⁺, K⁺-ATPase α1 and Na⁺, K⁺-ATPase α2 protein expression was decreased in Ryr1^{m1Nisw/+} adult muscle in low potassium and control diets when compared to Ryr1^{+/+} muscle on control potassium diet (FIG. 6a and FIGS. 6c-6e). Moreover, Na⁺, K⁺-ATPase α1 expression was decreased on the high potassium diet of Ryr1^{m1Nisw/+} and Ryr1^{+/+} muscle. The decreased expression suggests decreased Na⁺, K⁺-ATPase activity in these conditions. Strikingly, Na⁺, K⁺-ATPase α2 expression in Ryr1^{m1Nisw/+} muscle was restored to normal levels in high potassium diet (FIG. 6a and FIG. 6e), suggesting that Na⁺, K⁺-ATPase α2 may be a potential biomarker for CCD. Altogether, these data suggested that high potassium supplementation was able to rescue the levels of key proteins required for muscle activity in CCD muscle to similar levels as in RyR1^{+/+} mice. K_{ATP}6.1, Prkaa1, Abbc8, and Na⁺, K⁺-ATPase α1 and Na⁺, K⁺-ATPase α2 could be considered as potential biomarkers for CCD myopathy and recovery.

Biomarkers were further examined in RNA isolated from human skeletal muscles collected from CCD patients and compared to control human samples without muscle disease. Patients 1 and 2 had RYR1 mutations identified while patients 3 and 4, did not carry mutations in RYR1. As shown in Table 4, patients 1 and 2 showed a highly significant increase in expression of KCNJ8 (K_{ATP}6.1), PRKAA1, ABCC8 and ATPA1 (Na⁺, K⁺-ATPase α1). These data suggest that muscle from human CCD patients with RYR1 mutations is sensitive to pathways that mediate potassium homeostasis. Patient 3 had significantly increased expression of KCNJ8, PRKAA1 and ABCC8 but a decrease in ATPA1. In patient 4, only ABCC8 was elevated, whereas KCNJ8 and ATPA1 were significantly decreased. These data could suggest that differences in these potassium homeostasis pathways may lead to differential sensitivities in CCD patients with RYR1 mutations compared to those with non-RYR1 mediated CCD. These diagnostic biomarkers may eventually distinguish ideal patients for treatment of CCD through modulation potassium signaling pathways.

TABLE 4
Quantitative RT-PCR from muscle biopsies of four patients with CCD

| Patient | Ryr1 mutation | Kcnj8 | Prkaa1 | Abcc8 | Atpa1 |
|---|---|---|---|---|---|
| 1 | Yes | 2.16 +/− 0.07*** | 2.60 +/− 0.07*** | 92.03 +/− 3.45*** | 2.06 +/− 0.04*** |
| 2 | Yes | 1.40 +/− 0.02*** | 1.58 +/− 0.04*** | 33.39 +/− 1.48*** | 1.61 +/− 0.01*** |
| 3 | No | 2.07 +/− 0.05*** | 2.10 +/− 0.12*** | 4.98 +/− 0.05*** | 0.50 +/− 0.01\hat{ }\hat{ }\hat{ } |
| 4 | No | 0.72 +/− 0.01\hat{ }\hat{ }\hat{ } | 1.23 +/− 0.09 | 7.24 +/− 1.48*** | 0.33 +/− 0.01\hat{ }\hat{ }\hat{ } |

Values normalized to GAPDH before normalization to control human muscle RNA.
Asterisks and \hat{ } indicates significant value ***= 0.005 and \hat{ }\hat{ }\hat{ } = 0.005.

FIG. 6 represents exemplary data demonstrating altered expression of certain biomarkers involved in potassium transport, where FIG. 6a represents an exemplary Western blot of biomarkers Na⁺, K⁺-ATPase α2, and K_{ATP}6.1, with β-tubulin as a loading control; and FIGS. 6b-6e provide exemplary histograms of quantified protein levels of K_{ATP}6.1 (b), NKAα1 (c), NKAα2 (d) and pSer23/NKAα1 (e). Each sample was first normalized to its own loading control, and then the values from mutant and wild type from the same blot were compared. Statistical analyses were determined from several blots.

### Example 8

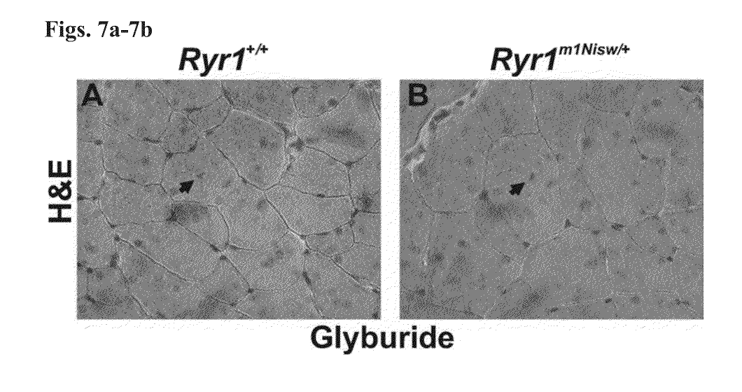

In certain exemplary methods, effect of a sulfonylurea class of anti-diabetic drugs on restoring membrane potential and muscle contractions in Ryr1^{m1Nisw/+} mice was examined. Muscle samples were isolated from 2-month-old Ryr1^{+/+} and Ryr1^{m1Nisw/+} mice administered a sulfonylurea drug, e.g. Glyburide (15 mg/kg/day oral gavage), which selectively binds SUR1/2 activating ATP-sensitive K⁺ channels. Increased grip and hanging strength was observed in Glyburide administered Ryr1^{m1Nisw/+} mice (FIGS. 5a-5c). Glyburide was also able to rescue the clinical and pathological manifestations of CCD in Ryr1^{m1Nisw/+} mice (FIGS. 7a-7b).

FIG. 7 represents an exemplary H&E staining results of cross-sections of vastus lateralis myofibers from Ryr1^{+/+} (A) and Ryr1^{m1Nisw/+} mice (B) treated with Glyburide. Ryr1^{m1Nisw/+} muscle demonstrated a significant reduction in the number of internal nuclei. Samples were from 2-month-old Ryr1^{+/+} and Ryr1^{m1Nisw/+} mice after one month gavage treatment with Glyburide (15 mg/kg/day).

### Example 9

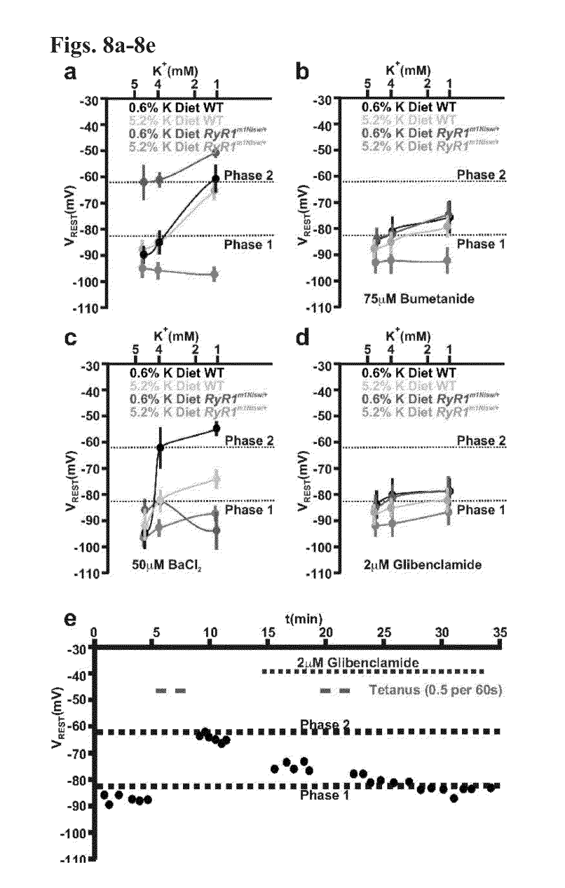

Skeletal muscles have two stable steady-state membrane potentials (V_{REST}) that diverge dependent on external concentrations of potassium and muscle fatigue. These states are caused by paradoxical depolarization due to a shift of serum potassium from the extracellular space into the myoplasm which depolarizes the muscle to a less-active state (˜60 mV; phase 2) from a normal more-active state (˜90 mV; phase 1, FIG. 8a, black (top) and light grey/yellow). Phase 2 increases the propensity for sodium channels to become inactive, leading to muscle failure and fatigue. In diseases with defects in ion channel conductance, muscles are depolarized and stuck in phase 2.

In wild type muscle exposed to low extracellular potassium (black line) and disease models (RyR1^{m1Nisw/+} soleus muscle; red line), muscles can be driven to phase 1 with bumetanide (75 μM), a drug which inhibits chloride conductance through the Na²⁺-K⁺-Cl⁺ cotransporter type two (NKCC2, FIG. 8b). Moreover in wild type muscle exposed to low extracellular potassium (black line) and disease models (RyR1^{m1Nisw/+} muscle; upper dark grey line/red), muscles can be driven to phase 2 by BaCl₂ (50 μM), an inhibitor of K_{IR} current (FIG. 8c). Chloride conductance is functionally linked to inward rectifying potassium channels. This suggests a role for K_{IR} current and chloride conductance in paradoxical depolarization during low K⁺ and muscle weakness disease. Even though multiple potassium inward rectifier channels are expressed in muscle, the best candidate for the K_{IR} current is the strongly rectifying K_{IR}2.1⁸.

In wild type two month old soleus muscle, paradoxical depolarization at normal (4.75 and 4.0 mM) and low (1.0 mM) extracellular potassium levels was similar to that suggested in the literature, as were the effects of bumetanide and Ba²⁺ on the active muscle state (FIGS. 8a-8 c; black and light grey/yellow lines). However in RyR1^{m1Nisw/+} soleus muscle, V_{REST} was continuously maintained at phase 2 (−62.2+/−5.2) independent of extracellular potassium concentrations (FIG. 8a; upper (top) dark grey line/red line). Chronic treatment with 5.2% potassium diet keeps the V_{REST} at phase 1, indicating that the physiology was rescued and suggesting that the defects were due to alterations in potassium homeostasis (FIG. 8a; blue line). Moreover in RyR1^{m1Nisw/+} soleus muscle on control potassium diets (0.6%), the addition of either bumetanide or BaCl₂ shifted V_{REST} to phase 1 (−92.3+/−4.8,−91.7+/−3.8 respectively) independent of extracellular potassium levels (FIGS. 8b-8c; black line). These data indicated that shifting the potassium homeostasis by diet or drug treatment rescued the muscle physiology.

In embryonic RyR1^{m1Nisw/m1Nisw} muscle, blockade of K_{IR} induced contractions with BaCl₂ and glybenclamide. In wild type adult muscle, glybenclamide (also known as glyburide) protects against fatigue caused by tetanic force. Glybenclamide (2 μM) blocks BaCl₂-sensitive K_{ATP}6.x channels. K_{ATP}6.x channels are activated through the increased accumulation of adenosine diphosphate (ADP) and decreased levels of adenosine triphosphate (ATP). This change in ADP/ATP ratios occurs during skeletal muscle fatigue. It was examined whether defects in potassium rectification were the cause of abnormal depolarization in the central core disease. Tetanus was applied to wild type muscle samples to cause a lower potassium condition. Addition of Glybenclamide protected against phase 2 shifting from phase 1 at the lower potassium concentrations (FIG. 8e) in wild type muscles and rescued the phase 2 V_{REST} to phase 1 in RyR1^{m1Nisw/+} soleus muscle (FIG. 8d; upper dark grey/red line). These data suggested that increased K_{ATP} channel activity was keeping RyR1^{m1Nisw/+} soleus muscle in a chronic state of fatigue, and a muscle intrinsic mechanism protected against muscle damage during fatigue and decreased ATP levels. These data also suggested that potassium rectifiers were important in the stabilization of phase 1 (K_{IR}2.1) and phase 2 (K_{ATP}6.x channels). Thus, the combination of an FDA approved drug, such as glybenclamide, and potassium supplementation may rescue the fatigue-like state of muscles in patients with CCD and other myopathies that lead to shift towards phase 2.

FIGS. 8a-8d represent exemplary graphs illustrating membrane potential shifts in RyR1^{+/+} and RyR1^{m1Nisw/+} muscles from mice on normal or high potassium diet elicited by various stimuli: reducing potassium concentration from 5 mM to 1 mM (a), and reducing potassium concentration in the presence of different drugs: 75 μM Bumetanide (b), 50 μM BaCl₂ (c), or 2 μM Glibenclamide (d). FIG. 8e provides an exemplary graph demonstrating membrane potential shift in RyR1^{+/+} muscle elicited by Tetanus, and effect of 2 μM Glibenclamide on Tetanus caused shift.

### Materials and Methods

### Strains and Forward Genetic Screen, Generating a CCD Mouse Model

ENU mutagenesis was performed as previously described on C57BL/6J background and then outcrossed onto 129S1/Svlmj background to score G3 embryos for recessive mutations that affect embryonic locomotion. E18.5 litters were dissected into cold Tyrode's solution containing 2.2 mM KCl and phenotyped for three types of locomotor movements; flexor-extension by pinching the paws, cross-extensor by holding the paws, and startle response by pinching the tail. A panel of 96 MIT and SKI SSLP markers mapped the Line1-4 mutation to the proximal third of chromosome 7. The genetic region containing the mutation was narrowed by the use of additional MIT SSLP markers on chromosome 7 and further meiotic mapping which followed linkage between the phenotype and C57BL/6J markers. As Ryr1 was a strong candidate gene in the narrowed 3 Mb interval, a complementation cross was performed between Ryr1^{m1Nisw/+} and RyR1^{tm1Alle} mice. These alleles did not complement as E18.5 trans-heterozygotes showed a similar non-motile phenotype as homozygotes of either allele. To identify the mutation in Line1-4, genomic DNA in overlapping segments of the Ryr1 gene were amplified by PCR from phenotypic E18.5 embryos and compared with control E18.5 C57Bl/6J DNA. All of the data presented here were obtained after outcrossing>8 generations onto 129S1/Svlmj background. Richard Allen generated the RyR1^{tm1Alle} (dyspedic) mice. In ˜5% of Ryr1^{tm1Alle/tm1Alle} embryos, exencephaly was documented, but none were used for experimentation.

### Immunohistochemistry

Diaphragms were dissected and pinned to sylgard dish in 4% paraformaldehyde for 10 min then permeabilized with 0.2% Triton/PBS for 30 min. Non-specific reactivity was blocked with 2% BSA/PBS for 2 hours. Primary monoclonal antibody against all RyRs (34C, 1:20) was applied overnight at room temperature. Secondary antibody (Alexa Fluor 488 conjugated donkey anti-mouse, Alexa Fluor 568-conjugated goat anti-mouse IgG, 1:500, Molecular Probes, #A11031) was applied for 1 hr. COX and NADH staining protocols are available at html://neuromuscular.wustl.edu/pathol/histol. Immunostained samples were examined by confocal laser scanning microscopy Zeiss LSM 510 META.

### Western Analysis

Muscle tissue from the limbs was dissected and washed in PBS and resuspended in RIPA buffer supplemented with Complete Mini Protease Inhibitor Cocktail (Roche) and Phosphatase Inhibitor Cocktail Set II (Calbiochem). Samples were sonicated for 1 minute (15 sec on, 15 sec off, 25% power) and incubated at 4° C. for 1 hour on a rocking platform. Samples were desalted with Zeba Spin Desalting Columns (7K MWCO, Thermo Scientific) and genomic DNA was digested with DNase I (Roche). Protein was quantified with the Bio-Rad Protein Assay (Bio-Rad). Samples to be probed for Erk, AMPK, and P-AMPK were incubated at 70° C. for 10 minutes prior to loading. All other samples were incubated at 37° C. for 30 minutes prior to loading. Protein was separated on 4-12% Bis-Tris gels (Invitrogen) and transferred to Immobilon-FL PVDF membranes (Millipore). Membranes were blocked in 5% dry milk in TBST for 1 hour. Primary antibody incubations were performed overnight at 4° C. while rocking Antibodies were diluted in TBST supplemented with 5% w/v BSA (Sigma). Secondary antibody incubations were performed for 1 hour at room temperature in TBST supplemented with 5% dry milk. Western blots were imaged using the Odyssey Infrared Imaging System (Li-Cor). Quantitation was performed using Odyssey v3.0 software (Li-Cor). Primary and secondary antibodies were used as follows: AMPKα (Cell Signaling, rabbit, 1:1000), Phospho-AMPKα (Cell Signaling, rabbit, 1:1000), AMPKβ1/2 (Cell Signaling, rabbit, 1:1000), Phospho-AMPKβ1 (Cell Signaling, rabbit, 1:1000), Erk1/2 (Cell Signaling, rabbit, 1:1000), Phospho-Erk (Cell Signaling, rabbit, 1:1000), KIR6.1 (Santa Cruz, goat, 1:200), KIR6.2 (Abcam, goat, 1:1000), KIR2.1 (Abcam, rabbit, 1:1000), Na/K-ATPase α1 (Cell Signaling, rabbit, 1:1000), Na/K-ATPase α2 (Millipore, rabbit, 1:1000), Phospho-Na/K ATPase α1 (Ser23, Cell Signaling, rabbit, 1:1000), Goat anti-Rabbit IRDye 680 (Li-Cor, 1:10000), Goat anti-Mouse IRDye 800CW (Li-Cor, 1:10000), Donkey anti-Goat IRDye 680LT (Li-Cor, 1:30000), Donkey anti-Mouse 800CW (Li-Cor, 1:10000).

### Quantitative RT-PC

RNA was extracted from limb muscle using Trizol followed by DNaseI digestion and clean-up (Qiagen RNAeasy Minikit) and reverse transcribed using random hexamer primers and SuperScript III Reverse Transcriptase (Invitrogen) and amplified using TaqMan Universal PCR Master Mix (Applied Biosystems). Quantitative PCR was performed on a Roche LightCycler 480 Real-Time PCR System. Calculations were performed by a relative standard curve method. Probes for target genes were from TaqMan Assay-on-Demand kits (Applied Biosystems). Samples were adjusted for total RNA content by TATA binding protein (TBP). Primers of use for methods disclosed herein can be derived by any methods known in the art.

### Potassium Diets

Diets were based on studies examining hypertension in mice. Diets were purchased through Harlan Laboratories, Inc. and are TD.10941 (0.1% K⁺), TD.10942 (0.6% K⁺), and TD.94121 (5.2% K⁺). These diets have similar percentages of three K⁺ sources.

### Calcium Transient Measurements

Intracellular Ca²⁺ was recorded with Fluo-3 (Invitrogen) at a final concentration of 200 μM that was injected by whole cell configuration, a waiting period of >5 min was used to allow the dye to diffuse into the cell interior. The total change in fluorescence (ΔF/F) was determined from the change in peak fluorescence from initial baseline during stimulation and F was the fluorescence immediately before the test pulse minus the measured average background (non Fluo-3) fluorescence before dye entry. For normal myotubes, average values of fluorescence change (ΔF) for each test potential (V) were fitted according to: (ΔF)=(ΔF)ₘₐₓ/{1+exp[(V_{F}−V)/k_{F}]}, where (ΔF)ₘₐₓ=1093 au, V_{F}=−1.4 mV and k_{F}=5.5 mV. Fluorescence emission was measured by fluorometer. Ryr1 cDNA was provided by Kurt Beam and Roger Bannister assisted in these experiments.

### Physiological Recordings

Sharp electrode recordings were performed as described. Dissected diaphragms were subject to recordings at room temperature in mouse Normal Ringers. For EMG recordings and contractions, preparation was warmed to 30° C. and continually superfused with oxygenated Tyrode's solution (2.2 mM KCl). EMG muscle recordings were made using fine-tip suction electrodes pulled from polyethylene tubing (PE-190; Clay Adams, NJ) and recorded via amplifiers (AI 401, Grass Amplifier connected with Digidata 1322a, Axon Instruments) directly on the computer with Axoscope 9 (Axoscope, CA). Significance of data was evaluated by Student's t test, which determined the p value (Excel 2008). A p value below 0.05 was considered significant.

Grip strength and Hanging Wire Task

To evaluate muscle weakness in vivo in RyR1^{m1Nisw/+} mice, we compared RyR1^{m1Nisw/+} and RyR1^{+/+} mice on the various diets or enalapril using grip strength tests and wire hanging task. Similar tests have been used to evaluate muscle weakness in CCD mice. Performance in Hanging Wire Task was scored on a 0 to 5 scale: 0, immediately fell off the bar; 1, hung onto bar with two forepaws; 2, hung onto bar with two forepaws and attempted to climb onto the bar; 3, hung onto the bar with two forepaws and one or both hind paws; 4, hung onto the bar with all four paws and tail wrapped around the bar; 5, hung onto the bar with all four paws and tail wrapped around the bar and escaped onto one of the supports. Malignant hyperthermia test was performed as previously described.

### Confocal Microscopy

Immunostained samples were examined by confocal laser scanning microscopy Zeiss LSM 510 META. Alexa-Fluor 568 was excited at 543-nm from a HeNe laser line (1 milliwatt maximum output, operated at 100%), directed via a 488/543 nm dual dichroic mirror. Emitted fluorescence was directed to a photomultiplier with a 560-nm long-pass filter. Confocal fluorescence intensity data were recorded as the average of four line scans per pixel and digitized at 8-bits, with photomultiplier gain adjusted such that maximum pixel intensities were <70% saturated.

All of the COMPOSITIONS and/or METHODS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. cm What is claimed is:

1. A method for treating a muscle myopathy disorder in a subject in need thereof comprising administering to the subject a therapeutically effective amount of a sulfonylurea agent or an agent that inhibits sulfonylurea receptor 1 (SUR1) and treating the muscle myopathy disorder in the subject.

2. The method of claim 1, wherein the sulfonylurea agent is selected from the group consisting of Carbutamide, Acetohexamide, Chlorpropamide, Tolbutamide, Tolazamide, Glipizide, Gliclazide, Glyburide (also known as Glibenclamide), Glibornuride,
Gliquidone, Glisoxepide, Glyclopyramide, and Glimepiride.

3. The method of claim 1, wherein the sulfonylurea agent is Glyburide.

4. The method of claim 1, wherein the sulfonylurea agent is administered orally to the subject.

5. The method of claim 4, wherein the sulfonylurea agent is administered at about 0.1 mg to about 10 mg.

6. The method of claim 1, wherein myopathy disorders comprise any muscle myopathy disorder, any congenital muscle myopathy disorder, central core disease (CCD), multi mini core disease, hypokalemic periodic paralysis, nemaline myopathy, myotubular myopathy, centronuclear myopathy, congenital fiber-type disproportion myopathy, hyaline body myopathy and malignant hyperthermia (MH).

7. The method of claim 1, wherein the muscle myopathy comprises central core disease (CCD).

8. A method for assessing presence of a muscle myopathy disorder in a subject comprising:
a. analyzing a muscle sample obtained from the subject for expression levels of one or more genes in the sample from the subject, wherein the one or more genes comprise one or more of Kcnj8, Prkaa1 Na+, K+-ATPase α1, Abcc8 and Na+, K+-ATPase α2;
b. comparing the molecular expression level(s) to molecular expression levels in a control subject; and
c. assessing presence of the muscle myopathy in the subject.

9. The method of claim 8, wherein the muscle myopathy disorders are associated with mutations of RyR1 (ryanodine receptor type 1).

10. The method of claim 8, wherein myopathy disorders comprise a muscle myopathy disorder, a congenital muscle myopathy disorder, central core disease (CCD), multi mini core disease, hypokalemic periodic paralysis, nemaline myopathy, myotubular myopathy, centronuclear myopathy, congenital fiber-type disproportion myopathy, hyaline body myopathy and malignant hyperthermia (MH).

11. The method of claim 8, wherein expression levels of Kcnj8, Na⁺, K⁺-ATPase α2 and Prkaa1 are increased compared to controls when the subject has a muscle myopathy disorder.

12. The method of claim 8, wherein expression levels of Kcnj8 and Na⁺, K⁺-ATPase α1 are decreased compared to controls when the subject has a muscle myopathy disorder.

13. The method of claim 8, wherein the subject is a human or non-human mammal.

14. The method of claim 8, wherein the subject is an infant or a fetus.

15. The method of claim 8, further comprising treating the subject having a muscle myopathy by administering a dietary potassium supplement to the subject.

16. The method of claim 8, further comprising treating the subject having a muscle myopathy by administering a sulfonylurea agent to the subject.

17. A method for treating a muscle myopathy disorder in a subject in need thereof, comprising, administering to the subject a therapeutically effective amount of a dietary supplement of potassium.

18. The method of claim 17, wherein the muscle myopathy disorders are associated with mutations of RyR1.

19. A dietary supplement composition comprising 0.1 mg to 10 mg of a sulfonylurea agent and 0.1 grams to 10 grams of a potassium agent.

20. The composition of claim 19, wherein the sulfonylurea agent comprises Glyburide.

21. The composition of claim 19, wherein the composition concentration of the agents are formulated for daily administration.

22. The composition of claim 19, wherein the sulfonylurea agent comprises Glimepiride.

23. A kit for diagnosing muscle myopathy disorders in a subject comprising:
one or more reagents to detect molecular expression levels of one or more genes of Kcnj8, Prkaa1, Na⁺, K⁺-ATPase α1 and Na⁺, K⁺-ATPase α2; and
one or more containers

24. The kit of claim 23, wherein the reagents for detecting molecular expression levels of the one or more genes include reagents for detecting mRNA levels of one or more genes.

25. The kit of claim 23, wherein the reagents for detecting molecular expression levels of the one or more genes include reagents for detecting protein levels of one or more genes.

26. A composition for treating central core disease (CCD) in a subject comprising:
potassium (K⁺) or a composition capable of releasing K⁺ from the cells of the subject; and
one or more agents known to treat CCD in a subject in need thereof and optionally, a pharmaceutically acceptable excipient.


SEQUENCE LISTING (4 sequences)

SEQ ID NO: 1 - DNA, 16 bp, Artificial
  derived from RyR1 gene
tttctgcgaggacacg

SEQ ID NO: 2 - protein, 5 aa, Artificial
  derived from RyR1 protein
FCEDT

SEQ ID NO: 3 - DNA, 16 bp, Artificial
  derived from mutant RyR1 gene
tttctgcggggacacg

SEQ ID NO: 4 - protein, 5 aa, Artificial
  derived from mutant RyR1 protein
FCGDT